(19) United States
(12) Patent Application Publication
(10) Pub. No.: US 2024/0417669 A1
(43) Pub. Date: Dec. 19, 2024

(54) SENSORS INCORPORATED INTO MATERIAL FOR MEDICAL DIAGNOSTICS

(71) Applicant: Lyten, Inc., San Jose, CA (US)

(72) Inventors: Sung Lim, Mountain View, CA (US); Daniel Cook, Woodside, CA (US); Jacques Nicole, Palo Alto, CA (US); Michael Stowell, Sunnyvale, CA (US); Ashley Lim, Mountain View, CA (US)

(21) Appl. No.: 18/814,320

(22) Filed: Aug. 23, 2024

Related U.S. Application Data
(63) Continuation-in-part of application No. 17/940,246, filed on Sep. 8, 2022.
Application No. 17/940,246 is a continuation-in-part of application No. 17/340,493, filed on Jun. 7, 2021, now Pat. No. 11,592,279.
Application No. 17/340,493 is a continuation-in-part of application No. 16/829,355, filed on Mar. 25, 2020, now Pat. No. 11,446,966.
Application No. 17/940,246 is a continuation-in-part of application No. 17/227,249, filed on Apr. 9, 2021, now Pat. No. 11,479,062.
Application No. 17/227,249 is a continuation-in-part of application No. 16/829,355, filed on Mar. 25, 2020, now Pat. No. 11,446,966.
(60) Provisional application No. 63/534,287, filed on Aug. 23, 2023.
(60) Provisional application No. 63/281,846, filed on Nov. 22, 2021.
(60) Provisional application No. 63/276,274, filed on Nov. 5, 2021.
(60) Provisional application No. 63/247,680, filed on Sep. 23, 2021.
(60) Provisional application No. 63/242,270, filed on Sep. 9, 2021.
(60) Provisional application No. 63/094,223, filed on Oct. 20, 2020.
(60) Provisional application No. 63/036,796, filed on Jun. 9, 2020.
(60) Provisional application No. 63/036,118, filed on Jun. 8, 2020.
(60) Provisional application No. 62/985,550, filed on Mar. 5, 2020.
(60) Provisional application No. 62/979,215, filed on Feb. 20, 2020.
(60) Provisional application No. 62/824,440, filed on Mar. 27, 2019.
(60) Provisional application No. 63/036,796, filed on Jun. 9, 2020.
(60) Provisional application No. 63/008,262, filed on Apr. 10, 2020.

(51) Int. Cl. C12M 1/34; C12Q 1/04

(52) U.S. Cl. CPC C12M 41/36; C12M 41/26; C12M 41/34; C12Q 1/04

(57) ABSTRACT

A resonant sensor is embedded within or applied to a component of a medical diagnostic apparatus. The resonant sensor is formed from a composite material. The resonant sensor undergoes a change of permittivity and/or change in permeability due to metabolic activity of a microorganism that is involved in the medical diagnostic and proximal to the resonant sensor. The medical diagnostic apparatus may be a blood culture bottle that is configured to contain a blood culture medium. The resonant sensor may be embedded in or applied to the exterior or interior wall of the blood culture bottle. The resonant sensor may undergo a change in permittivity and/or a change in permeability due to production of carbon dioxide by the microorganism. The composite material may comprise a carbonaceous material such as graphene.

### CROSS-REFERENCE TO RELATED APPLICATIONS

This patent application claims the benefit of priority to U.S. Provisional Patent Application No. 63/534,287, entitled “SENSORS INCORPORATED INTO MATERIAL FOR MEDICAL DIAGNOSTICS,” filed Aug. 23, 2023, which is assigned to the assignee hereof; the disclosure of the prior application is considered part of and is incorporated by reference in this patent application.

This patent application is a continuation-in-part of and claims the benefit of priority to U.S. patent application Ser. No. 17/940,246, entitled “SENSORS INCORPORATED INTO BUILDING MATERIALS TO DETECT PHYSICAL CHARACTERISTIC CHANGES,” filed Sep. 8, 2022, which in turn claims the benefit of priority to: U.S. Provisional Patent Application No. 63/242,270, entitled “SENSORS INCORPORATED INTO SEMI-RIGID STRUCTURAL MEMBERS TO DETECT PHYSICAL CHARACTERISTIC CHANGES” filed Sep. 9, 2021; U.S. Provisional Patent Application No. 63/247,680, entitled “SENSORS INCORPORATED INTO SEMI-RIGID STRUCTURAL MEMBERS TO DETECT PHYSICAL CHARACTERISTIC CHANGES” and filed Sep. 23, 2021; U.S. Provisional Patent Application No. 63/276,274, entitled “SENSORS INCORPORATED IN VEHICLE COMPONENTS TO DETECT PHYSICAL CHARACTERISTIC CHANGES, and filed Nov. 5, 2021; and U.S. Provisional Patent Application No. 63/281,846, entitled “SENSORS INCORPORATED INTO AIRBORNE VEHICLE COMPONENTS TO DETECT PHYSICAL CHARACTERISTIC CHANGES” and filed Nov. 22, 2021, all of which are assigned to the assignee hereof; the disclosures of all prior applications are considered part of and are incorporated by reference in this patent application.

U.S. patent application Ser. No. 17/940,246 is also a continuation-in-part of and claims the benefit of priority to U.S. patent application Ser. No. 17/227,249, entitled “TUNED RADIO FREQUENCY (RF) RESONANT MATERIALS AND MATERIAL CONFIGURATIONS FOR SENSING IN A VEHICLE” and filed on Apr. 9, 2021, which in turn, claims the benefit of priority to U.S. Provisional Patent Application No. 63/008,262, entitled “RESONANCE SENSING IN TIRES” and filed on Apr. 10, 2020, and to U.S. Provisional Patent Application No. 63/036,796, entitled “RESONANCE SENSING IN ELASTOMER-CONTAINING PRODUCTS” and filed on Jun. 9, 2020, all of which are assigned to the assignee hereof; the disclosures of all prior applications are considered part of and are incorporated by reference in this patent application.

U.S. patent application Ser. No. 17/227,249 is also a continuation-in-part of and claims the benefit of priority to U.S. patent application Ser. No. 16/829,355, entitled “TIRES CONTAINING RESONATING CARBON-BASED MICROSTRUCTURES” and filed on Mar. 25, 2020, which in turn, claims the benefit of priority to U.S. Provisional Patent Application No. 62/985,550, entitled “RESONANT SERIAL NUMBER IN VEHICLE TIRES” and filed on Mar. 5, 2020, to U.S. Provisional Patent Application No. 62/979,215, entitled “WASTE ENERGY HARVESTING AND POWERING IN VEHICLES” and filed on Feb. 20, 2020, and to U.S. Provisional Patent Application No. 62/824,440, entitled “TUNING RESONANT MATERIALS FOR VEHICLE SENSING” and filed on Mar. 27, 2019, all of which are assigned to the assignee hereof; the disclosures of all prior applications are considered part of and are incorporated by reference in this patent application.

U.S. patent application Ser. No. 17/940,246 is a continuation-in-part of and claims the benefit of priority to U.S. patent application Ser. No. 17/340,493, entitled “SENSORS INCORPORATED INTO ELASTOMERIC MATERIALS TO DETECT ENVIRONMENTALLY-CAUSED PHYSICAL CHARACTERISTIC CHANGES” and filed on Jun. 7, 2021, which in turn, claims the benefit of priority to U.S. Provisional Patent Application No. 63/036,118, entitled “CARBON-CONTAINING STICTION SENSORS” and filed on Jun. 8, 2020, to U.S. Provisional Patent Application No. 63/094,223, entitled “SENSORS FOR ELASTOMER PROPERTY CHANGE DETECTION” and filed on Oct. 20, 2020, and to, U.S. Provisional Patent Application No. 63/036,796, entitled “RESONANCE SENSING IN ELASTOMER-CONTAINING PRODUCTS” and filed on Jun. 9, 2020, all of which are assigned to the assignee hereof; the disclosures of all prior applications are considered part of and are incorporated by reference in this patent application.

U.S. patent application Ser. No. 17/340,493 is also a continuation-in-part of and claims the benefit of priority to U.S. patent application Ser. No. 16/829,355, entitled “TIRES CONTAINING RESONATING CARBON-BASED MICROSTRUCTURES” and filed on Mar. 25, 2020, which in turn, claims the benefit of priority to U.S. Provisional Patent Application No. 62/824,440, entitled “TUNING RESONANT MATERIALS FOR VEHICLE SENSING” and filed on Mar. 27, 2019, all of which are assigned to the assignee hereof; the disclosures of all prior applications are considered part of and are incorporated by reference in this patent application.

### TECHNICAL FIELD

The present disclosure relates to sensors incorporated into materials for medical diagnostics, and more particularly to split-ring resonators embedded in materials for detecting microorganism growth and antimicrobial susceptibility.

### DESCRIPTION OF RELATED ART

Rapid and accurate detection of microorganisms is crucial in various fields, including medical diagnostics, food safety, and environmental monitoring. Traditional methods for detecting and identifying microorganisms often rely on time-consuming culture-based techniques that can take days to yield results. This delay can have significant consequences, particularly in healthcare settings where prompt diagnosis and treatment of infections are essential for patient outcomes.

In recent years, there has been growing interest in developing faster and more sensitive methods for microorganism detection. Various approaches have been explored, including molecular techniques, immunoassays, and biosensors. However, many of these methods still face challenges related to complexity, cost, or the need for specialized equipment and trained personnel.

As such, there is thus a need for addressing these and/or other issues associated with the prior art.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

In some aspects, the techniques described herein relates to a resonant sensor, wherein the resonant sensor undergoes a change of permittivity and/or a change of permeability due to metabolic activity of a proximal microorganism.

In some aspects, the techniques described herein relate to a component, including: a material; and at least one split-ring resonator (SRR) embedded within the material, wherein the at least one SRR is formed from a composite material, wherein the at least SRR undergoes a change of permittivity and/or permeability due to metabolic activity of a microorganism.

In some aspects, the techniques described herein relate to a component, wherein the material is a blood culture medium.

In some aspects, the techniques described herein relate to a component, wherein the blood culture medium is contained within a blood culture bottle.

In some aspects, the techniques described herein relate to a component, wherein the at least one SRR is embedded within a wall of the blood culture bottle or the at least one SRR is embedded in an adhesive or label affixed to an exterior wall of the blood culture bottle.

In some aspects, the techniques described herein relate to a component, wherein the change in permittivity is due to production of carbon dioxide by the microorganism.

In some aspects, the techniques described herein relate to a component, wherein the composite material includes a carbonaceous material.

In some aspects, the techniques described herein relate to a component, wherein the carbonaceous material includes graphene.

In some aspects, the techniques described herein relate to a component, further including a processor configured to analyze the change in permittivity to determine antimicrobial susceptibility of the microorganism.

In some aspects, the techniques described herein relate to a component, wherein the processor is further configured to generate a report indicating antimicrobial susceptibility results.

In some aspects, the techniques described herein relate to a component, wherein the at least one SRR is configured to resonate at a first frequency in response to an electromagnetic stimulus when the material is in a first state.

In some aspects, the techniques described herein relate to a component, wherein the at least one SRR is configured to resonate at a second frequency in response to the electromagnetic stimulus when the material is in a second state.

In some aspects, the techniques described herein relate to a component, wherein the first state corresponds to an absence of the microorganism and the second state corresponds to a presence of the microorganism.

In some aspects, the techniques described herein relate to a component, wherein the at least one SRR includes a plurality of SRRs arranged in an array.

In some aspects, the techniques described herein relate to a component, wherein each SRR in the array is configured to detect changes in permittivity in a different region of the material.

In some aspects, the techniques described herein relate to a component, further including an electromagnetic stimulus source configured to emit an electromagnetic signal to stimulate the at least one SRR.

In some aspects, the techniques described herein relate to a component, wherein the electromagnetic stimulus source is configured to emit a chirp signal spanning a range of frequencies.

In some aspects, the techniques described herein relate to a component, further including a detector configured to measure changes in at least one of amplitude and frequency of a resonant response of the at least one SRR.

In some aspects, the techniques described herein relate to a component, wherein the at least one SRR is configured to detect changes in permittivity due to changes in pH of the material.

In some aspects, the techniques described herein relate to a component, wherein the component is part of an antimicrobial susceptibility testing system including multiple wells, each well containing a different antimicrobial agent.

In some aspects, the techniques described herein relate to a component, wherein the at least one SRR is configured to detect changes in permittivity in each well to determine effectiveness of different antimicrobial agents against the microorganism.

A disclosed apparatus includes sensors incorporated into material. In use, a component may include at least one split-ring resonator (SRR) embedded within a material of the component, wherein the at least one SRR is formed from a composite material. Additionally, the at least SRR undergoes a change of permittivity due to metabolic activity of a microorganism.

A disclosed apparatus includes sensors incorporated into material. In use, a component may include at least one split-ring resonator (SRR) embedded within a material of the component, wherein the at least one SRR is formed from a composite material. Additionally, the at least SRR undergoes a change of permeability due to metabolic activity of a microorganism.

Details of one or more implementations of the subject matter described in this disclosure are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1 illustrates a process flow for analyzing bacterial cultures using resonant sensor technology, according to aspects of the present disclosure.

FIG. 2 depicts views of vials with resonant sensors for detecting bacterial growth, according to an embodiment.

FIG. 3 illustrates views of a blood culture bottle system for bacterial detection, according to aspects of the present disclosure.

FIG. 4 depicts a system diagram of a rapid antimicrobial susceptibility testing system, according to an embodiment.

FIG. 5 illustrates a diagram of applicability of a rapid antimicrobial susceptibility testing system, according to aspects of the present disclosure.

FIG. 6 depicts a comparison of conventional and resonant sensor enabled antimicrobial susceptibility testing systems, according to an embodiment.

FIG. 7A through FIG. 7Y depict carbonaceous materials used as a formative material to produce any of the presently disclosed resonators (e.g., split ring resonators), in accordance with one embodiment.

Like reference numbers and designations in the various drawings indicate like elements.

### DETAILED DESCRIPTION

Sepsis is a serious medical condition characterized by the presence of pathogenic agents in the bloodstream, which triggers a systemic inflammatory response throughout the body. For example, across the years 1979 to 2000, approximately 750 million hospitalizations in the United States, with around 10 million identified cases of sepsis. The number of sepsis cases showed an annual increase of 8.7%, starting from 164,000 cases in 1979 and reaching 660,000 cases in 2000. Furthermore, it is projected that the incidence of sepsis will outpace the population growth rate in the future.

In other studies, the incidence rate of sepsis was reported as 3.0 cases per 1000 individuals and 2.26 cases per 100 hospital discharges. Out of the 192,980 identified cases, 51.5% received intensive care, and an additional 17.3% required ventilation in an intermediate care unit. Notably, the incidence rate of sepsis exhibited a significant rise with advancing age, ranging from 0.2 cases per 1000 children to 26.2 cases per 1000 elderly patients aged over 85 years old. This trend raises substantial concerns for healthcare, particularly as the aging population of the United States continues to grow.

Rapid bacterial detection is crucial for sepsis diagnosis due to a life-threatening condition of sepsis that requires immediate medical intervention. Once the presence and identification of etiologic bacteria have been determined, an AST (antimicrobial susceptibility test) can be performed to select targeted antibiotic therapy. If the blood stream infection is suspected, healthcare professionals can initiate broad spectrum antibiotic therapy, which can be followed by tailored treatment once the AST results are available.

In the present disclosure, passive resonant sensors present a promising solution for monitoring bacterial growth in various blood culture media. Unlike conventional methods, this novel approach eliminates the need for an additional manufacturing step of integrating an indicator into the blood culture bottle (in one embodiment). Such an approach not only reduces manufacturing costs but may also help in preventing potential contamination issues. Moreover, the highly sensitive resonant sensor provides an opportunity to detect bacterial growth faster compared to traditional methods (such as those that rely on CO2 or O2 detection). By leveraging this advanced technology, the detection of bacteria can be expedited, enabling prompt intervention and timely treatment. As such, the passive resonant sensor may offer advantages such as cost-effectiveness, avoidance of contamination risks, and faster detection of bacterial growth in blood culture media, making it a promising tool for enhancing sepsis diagnosis and improving patient outcomes.

The present disclosure provides systems, methods, and components for detecting microorganism growth and determining antimicrobial susceptibility. These systems, methods, and components may incorporate resonant sensors and/or split-ring resonators (SRRs) into materials, such as blood culture media or antimicrobial susceptibility test panels. The sensors, which may be formed from composite materials, may be configured to detect changes in permittivity due to the metabolic activity of microorganisms. This approach offers a potential solution for rapid and cost-effective detection of microorganism growth and determination of antimicrobial susceptibility, addressing the limitations of conventional methods that often require time-consuming culture-based techniques. The disclosed technology may find potential applications in various fields, including medical diagnostics, food safety, and environmental monitoring, among others.

In some cases, the resonant sensors may be embedded within a material of a component, such as a blood culture bottle or an antimicrobial susceptibility test panel. In other cases, the resonant sensors may be disposed within or adjacent to each well of a test panel. The resonant sensors may be formed from various composite materials, including carbonaceous materials such as graphene. The resonant sensors may be configured to detect changes in permittivity due to various factors, such as the production of carbon dioxide by the microorganisms or changes in the dielectric constant of the growth media. The disclosed technology thus provides a versatile and efficient approach for detecting microorganism growth and determining antimicrobial susceptibility.

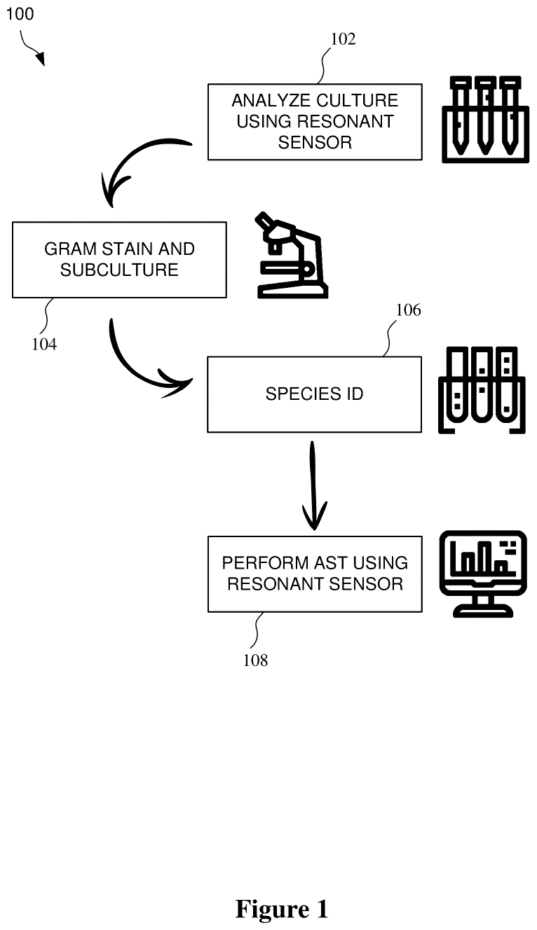

FIG. 1 illustrates a process flow 100 for analyzing bacterial cultures using resonant sensor technology. As an option, the process flow 100 may be implemented in the context of any one or more of the embodiments set forth in any previous and/or subsequent figure(s) and/or description thereof. Of course, however, the process flow 100 may be implemented in the context of any desired environment. Further, the aforementioned definitions may equally apply to the description below.

As shown, the process flow 100 begins with a culture analysis step 102, where a culture is analyzed using a resonant sensor. The resonant sensor may be embedded within a material of a component (including but not limited to a vial, a bottle, a sample, etc.), such as a blood culture medium, on the exterior of the component, and/or on an apparatus which may hold the component. The resonant sensor may be formed from a composite material, such as a carbonaceous material, and may be configured to detect changes in permittivity due to the metabolic activity of a microorganism.

In some cases, the resonant sensor may be configured to detect changes in permittivity due to the production of carbon dioxide by the microorganism. Such changes in the concentration of carbon dioxide in the culture medium may indicate the growth of the microorganism.

Following the initial culture analysis step 102, the process flow 100 moves to a gram stain and subculture step 104. This step may involve staining the culture with a gram stain to differentiate between Gram-positive and Gram-negative bacteria, and subculturing the bacteria to isolate individual colonies for further analysis.

With a Gram stain, a bacterial sample may be first stained with crystal violet dye, followed by iodine, which forms a complex with the dye. The sample may then be decolorized with alcohol or acetone, and counterstained with safranin. Gram-positive bacteria retain the crystal violet-iodine complex and appear purple, while Gram-negative bacteria take up the safranin and appear pink. Following this microscopic examination, the sample may be subcultured by transferring a portion onto an appropriate growth medium. Such a medium may allow the bacteria to grow into isolated colonies, which can be further analyzed to identify the bacterial species and determine their susceptibility to antibiotics.

The next step in the process flow 100 is a species identification step 106. This step may involve various laboratory techniques to identify the specific species of bacteria present in the culture.

The final step in the process flow 100 is an antimicrobial susceptibility testing (AST) performance step 108 using a resonant sensor. This step may involve exposing the bacteria to various antibiotics and monitoring the changes in permittivity detected by the resonant sensor to determine the susceptibility of the bacteria to the antibiotics. The changes in permittivity may reflect the changes in the metabolic activity of the bacteria in response to the antibiotics, providing a rapid and accurate measure of antibiotic susceptibility.

In some aspects, the process flow 100 may be automated, with the resonant sensor providing real-time data on bacterial growth and antibiotic susceptibility. This may enable rapid and accurate diagnosis of bacterial infections and determination of appropriate antibiotic treatment, potentially improving patient outcomes.

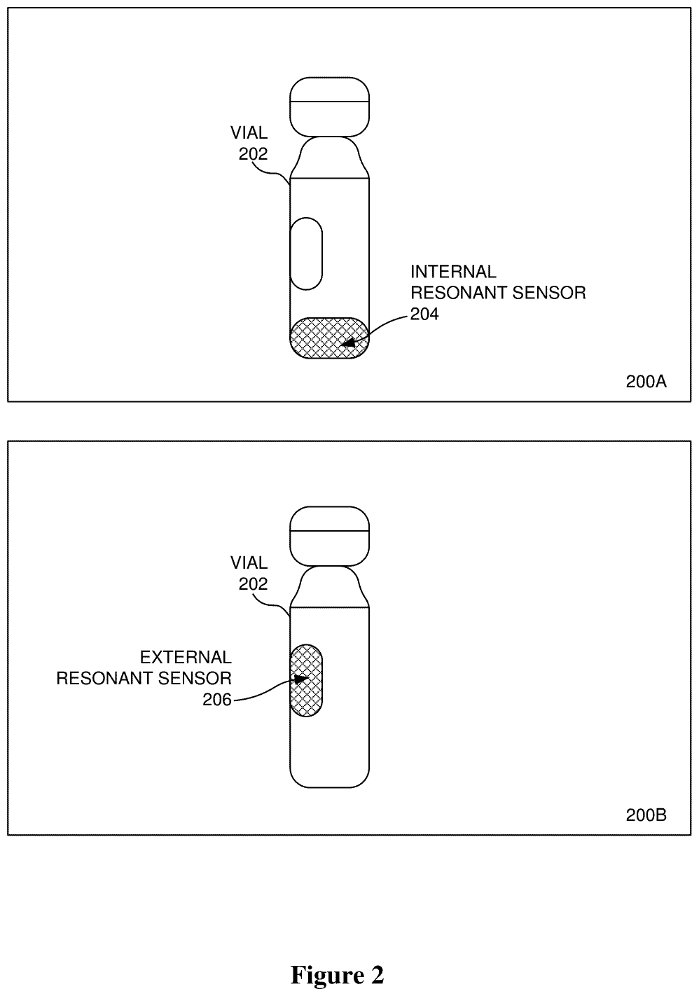

FIG. 2 depicts views 200A and 200B of vials with resonant sensors for detecting bacterial growth, according to an embodiment. As an option, the views 200A and 200B may be implemented in the context of any one or more of the embodiments set forth in any previous and/or subsequent figure(s) and/or description thereof. Of course, however, the views 200A and 200B may be implemented in the context of any desired environment. Further, the aforementioned definitions may equally apply to the description below.

As shown, the first configuration of view 200A features a vial 202 with an internal resonant sensor 204. The internal resonant sensor 204 may be embedded within the material of the vial 202, such as at the bottom of the vial. It is recognized that the internal resonant sensor 204 may be found at any location internal to the vial 202. Such a placement may allow the internal resonant sensor 204 to be in direct contact with the blood culture medium contained within the vial. The placement of the internal resonant sensor may allow detection of changes in permittivity due to the metabolic activity of microorganisms.

In the second configuration, shown in view 200B, the vial 202 is equipped with an external resonant sensor 206. The external resonant sensor 206 is disposed on the outer surface of the vial 202. Despite being outside the vial, the external resonant sensor 206 is still capable of detecting changes in permittivity due to the metabolic activity of microorganisms. This is because the resonant sensor can detect changes in the dielectric properties of the material of the vial, which can be influenced by changes in the blood culture medium inside the vial.

In some cases, the internal resonant sensor 204 and the external resonant sensor 206 may be formed from a composite material, such as a carbonaceous material. The composite material may be selected for its desirable properties, such as high sensitivity to changes in permittivity. The internal resonant sensor 204 and the external resonant sensor 206 may be configured to detect changes in permittivity due to various factors, such as the production of carbon dioxide by the microorganisms or changes in the dielectric constant of the growth media. In other embodiments, metabolic VOCs (and other metabolites) may be generated by the media (which may be measured by carbon dioxide), which in turn may change the pH within the vial, which in turn may be correlated to permittivity. It is to be appreciated that other gases may be generated within the vial, which may be likewise detected (and correlated to a change in permittivity).

The choice between internal and external resonant sensors may depend on various factors, such as the specific requirements of the blood culture analysis, the properties of the blood culture medium, and the design constraints of the vial and the resonant sensor. For instance, an internal resonant sensor may be preferred for its potentially higher sensitivity, while an external resonant sensor may be favored for its case of implementation and reduced risk of contamination. In some aspects, both internal and external resonant sensors may be used in combination to provide a more comprehensive analysis of the blood culture.

It is recognized that the exterior resonant sensor 206 may be in the form of a printed label, an adhesive, etc., consistent with the disclosure herein.

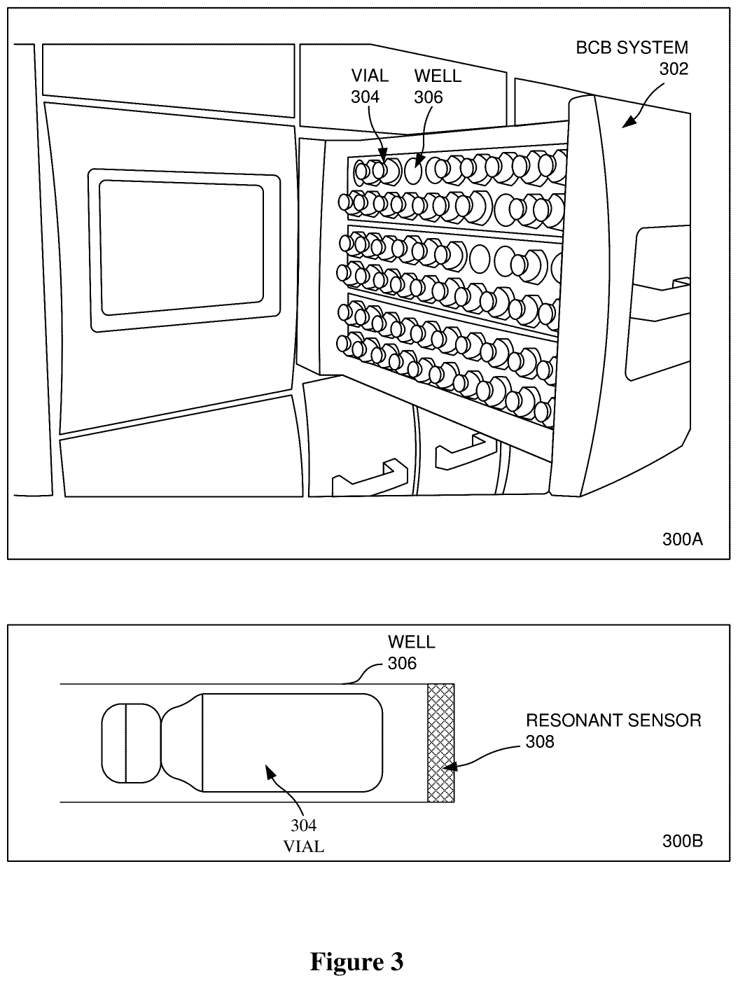

FIG. 3 illustrates views 300A and 300B of a blood culture bottle system for bacterial detection, according to aspects of the present disclosure. As an option, the views 300A and 300B may be implemented in the context of any one or more of the embodiments set forth in any previous and/or subsequent figure(s) and/or description thereof. Of course, however, the views 300A and 300B may be implemented in the context of any desired environment. Further, the aforementioned definitions may equally apply to the description below.

As shown, the view 300A shows a blood culture bottle (BCB) system 302 for bacterial detection. The BCB system 302 comprises multiple wells 306 arranged in a grid pattern. Each well 306 is designed to hold a vial 304 containing a blood sample for culturing.

In some aspects, the vial 304 may contain a blood culture medium. The blood culture medium may be a liquid medium designed to promote the growth of bacteria from the blood sample. The blood culture medium may contain various nutrients and other substances that support bacterial growth. In some cases, the blood culture medium may be specifically formulated to promote the growth of certain types of bacteria, such as aerobic or anaerobic bacteria.

In some cases, the BCB system 302 may incorporate resonant sensors for detecting bacterial growth in the blood samples. The resonant sensors may be embedded within a material of the component, such as the blood culture medium contained within the vial 304. The resonant sensors may be formed from a composite material, such as a carbonaceous material. The resonant sensors may be configured to detect changes in permittivity due to the metabolic activity of a microorganism.

In various embodiments, and in one particular example, blood culturing may be used to identify the specific bacteria responsible for sepsis. The process involves drawing a blood sample from the patient, which is then placed in a blood culture bottle (BCB). The BCB may be equipped with a colorimetric or fluorogenic indicator that is sensitive to CO2, a metabolic byproduct released by bacteria during growth. The sample is incubated at 37° C., allowing any present bacteria to proliferate. As the bacterial population expands, it releases increasing amounts of CO2, leading to a noticeable change in the indicator's color. This color change is interpreted as a positive test result, indicating the presence of bacteria. If a blood culture bottle fails to show a positive result within 5 days, it is considered a negative test for bacterial growth. To accommodate the high demand, large-scale automated systems for BCB incubation and monitoring have been developed. These systems are modularized to facilitate scalability and have the capacity to handle hundreds of blood cultures per day. However, the present novel blood culture system includes resonant sensors may be used to rapidly detect the presence of bacteria in both aerobic and anerobic blood culture bottles.

The view 300B provides a detailed look at an individual well 306 and vial 304 configuration. The vial 304 is shown inserted into the well 306. At the bottom of the well 306, a resonant sensor 308 is positioned within the well cavity. This resonant sensor 308 is designed to detect changes in the sample's properties, potentially indicating bacterial growth. In some aspects, the resonant sensor 308 may be embedded within a wall of the well 306.

The BCB system 302 integrates multiple components to facilitate automated blood culture analysis. The arrangement of wells 306 allows for simultaneous processing of numerous samples. The inclusion of the resonant sensor 308 within each well cavity enables real-time monitoring of the blood samples for bacterial growth detection.

It is to be appreciated that the resonant sensor 308 may be implemented within the BCB system 302 in a variety of configurations. For example, the resonant sensor 308 may be located within each well 306 of the BCB system 302. In an alternative configuration (not shown), a single (or any preconfigured number) resonant sensor may be attached to a x-y axis robot within the BCB system 302 which can then be axially moved to measure vial(s) of the BCB system 302. Further, with respect to the views 200A and 200B where the resonant sensor may be applied internally or externally to the vial, the BCB system may be further configured (not shown) to wireless interrogate one or more of the resonant sensors located in/on each vial.

In this manner, the BCB system 302 may be configured with resonant sensors in one or more wells, with a resonant sensor attached to a x-y axis robot, and/or with an interrogator to wireless interrogate any resonant sensor located on/in a vial and/or within the BCB system 302.

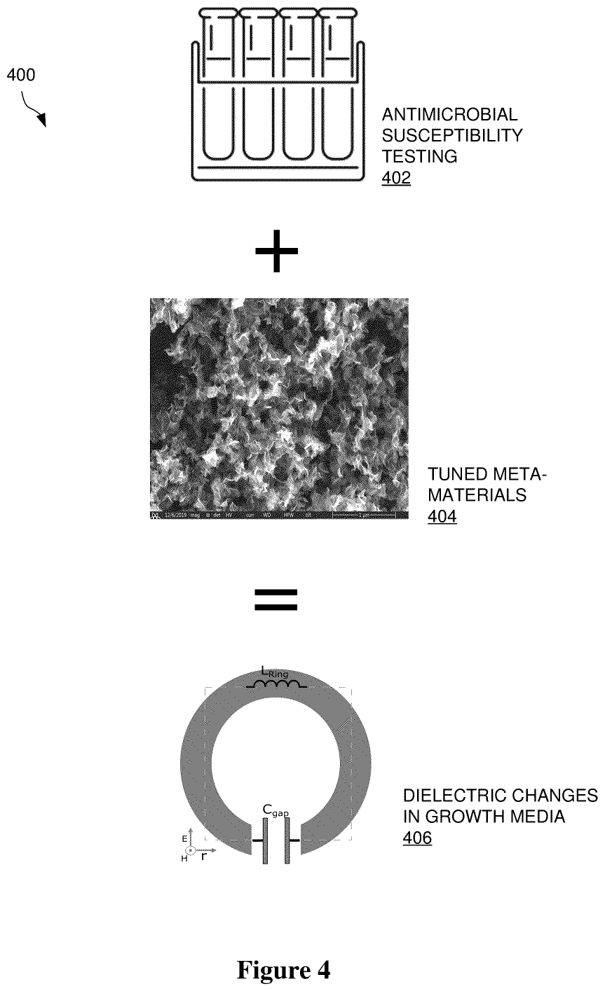

FIG. 4 depicts a system diagram 400 of a rapid antimicrobial susceptibility testing system, according to an embodiment. As an option, the system diagram 400 may be implemented in the context of any one or more of the embodiments set forth in any previous and/or subsequent figure(s) and/or description thereof. Of course, however, the system diagram 400 may be implemented in the context of any desired environment. Further, the aforementioned definitions may equally apply to the description below.

As shown, the system diagram 400 comprises three main components: an antimicrobial susceptibility testing panel 402, tuned meta-materials 404, and a dielectric changes detector 406.

The antimicrobial susceptibility testing panel 402 is shown as the first step. The sample may be a blood culture medium or any other suitable medium for bacterial growth.

As a second step, the tuned meta-materials 404 represent carbonaceous material engineered to have specific electromagnetic properties that can be used for sensing applications. In some aspects, the meta-materials 404 may be tuned to detect changes in permittivity due to the metabolic activity of microorganisms. In some cases, at least one resonant sensor (and/or split-ring resonator (SRR), etc.) may be disposed within or adjacent to each well of the test panel. The resonant sensor may be formed from a composite material, such as a carbonaceous material, and may be configured to detect changes in permittivity, such as the production of carbon dioxide by the microorganisms or changes in the dielectric constant of the growth media.

The combination of the antimicrobial susceptibility testing panel 402 and the tuned meta-materials 404 may result in dielectric changes by detector 406, which may be configured to detect changes in the dielectric properties of the growth media, which can indicate bacterial growth or inhibition in response to antibiotics. In some cases, the detector 406 may be configured to measure changes in permittivity of the meta-material (such as within a resonant sensor and/or SRR). The changes in permittivity may reflect the changes in the metabolic activity of the bacteria in response to the antibiotics, providing a rapid and accurate measure of antibiotic susceptibility.

In particular, the system diagram 400 shows an unobvious combination of using a typical AST assembly but combining it with a meta-material, such as a resonant sensor, may then provide enhanced sensing, including measuring changes in the bacterial samples when exposed to different antibiotics. This combination allows for faster and potentially more sensitive detection of antibiotic effectiveness compared to traditional AST methods.

It is to be appreciated that the system diagram may also include a processor (not shown) configured to analyze the changes in permittivity detected by the resonant sensor to determine antimicrobial susceptibility of the microorganisms. The processor may be a dedicated hardware component, a software module running on a general-purpose computing device, or a combination thereof. In some cases, the processor may be integrated with the resonant sensor, the antimicrobial susceptibility testing panel 402, the tuned meta-materials 404, the detector 406, and/or other components of the system diagram 400. In other cases, the processor may be a separate component that communicates with the resonant sensor and other components of the system diagram 400 via wired or wireless connections.

The processor may be configured to analyze the changes in permittivity based on various factors, such as the magnitude of the changes, the rate of the changes, the pattern of the changes, and the correlation of the changes with the concentrations of different antibiotics via the antimicrobial susceptibility testing panel 402. The processor may use various data analysis techniques, such as statistical analysis, machine learning, pattern recognition, or other suitable techniques, to analyze the changes in permittivity and determine antimicrobial susceptibility.

In some aspects, the processor may be further configured to generate a report indicating antimicrobial susceptibility results for different antimicrobial agents tested in the multiple wells of the antimicrobial susceptibility testing panel 402. The report may include various types of information, such as the identities of the antimicrobial agents, the concentrations of the antimicrobial agents, the changes in permittivity detected by the resonant sensor for each antimicrobial agent, and the determined antimicrobial susceptibility for each antimicrobial agent.

The resonant sensor in the rapid AST system may be configured to detect a change in permittivity due to metabolic activity of a microorganism. For example, the metabolic activity of a microorganism may include various processes, such as respiration, fermentation, biosynthesis, and other processes, that may cause changes in the composition, properties, or conditions of the growth media. These changes may result in changes in the permittivity of the growth media, which can be detected as dielectric changes in the growth media (via detector 406).

In some cases, the resonant sensor is configured to detect a change in permittivity due to production of carbon dioxide by the microorganism. The resonant sensor may be tuned to be sensitive to the changes in permittivity caused by the production of carbon dioxide, which in turn may cause a change in dielectric properties of the growth media (measured by the detector 406). It is recognized that the changes in the dielectric properties of the growth media may include the permittivity, the dielectric constant, the dielectric loss, or other dielectric properties. The detector 406 may provide additional information or confirmation about the changes in permittivity detected by the resonant sensor, and may enhance the accuracy, reliability, or other aspects of the antimicrobial susceptibility testing.

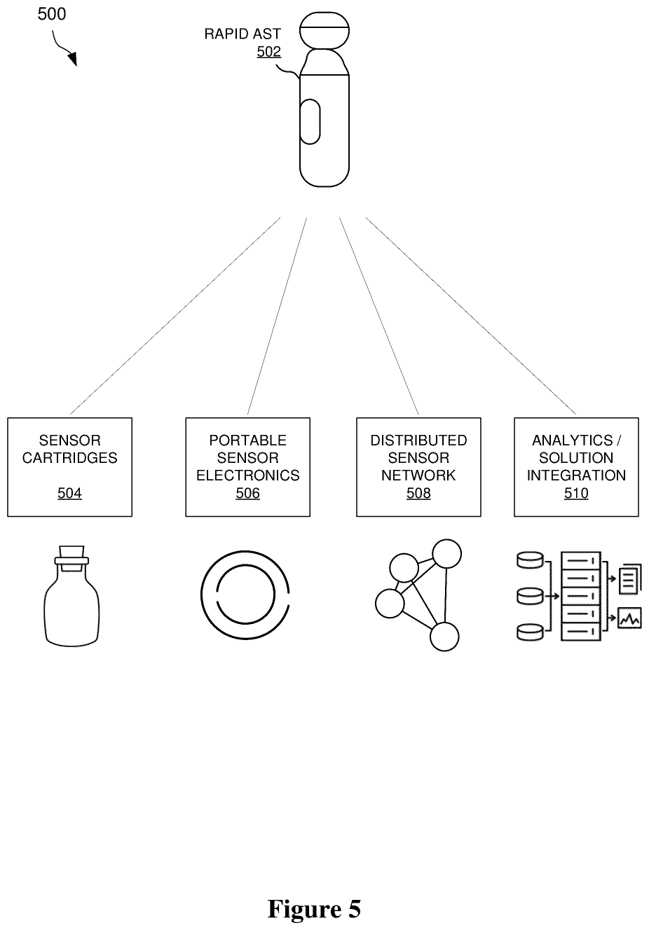

FIG. 5 illustrates a diagram 500 of applicability of a rapid antimicrobial susceptibility testing system, according to aspects of the present disclosure. As an option, the diagram 500 may be implemented in the context of any one or more of the embodiments set forth in any previous and/or subsequent figure(s) and/or description thereof. Of course, however, the diagram 500 may be implemented in the context of any desired environment. Further, the aforementioned definitions may equally apply to the description below.

As shown, the diagram 500 comprises a rapid AST 502, which is connected to four main components, including sensor cartridges 504, portable sensor electronics 506, distributed sensor network 508, and/or analytics/solution integration 510. For example, the diagram 500 shows the applicability of the rapid AST 502 to various fields and markets. Additionally, within the context of the present description, rapid AST 502 refers to an AST with a resonant sensor.

The sensor cartridge 504 represents that a resonant sensor can be configured within a cartridge, vial, and/or bottle. As discussed herein, such a resonant sensor may be configured on the exterior, and/or interior of the cartridge (see, e.g. FIG. 2).

The portable sensor electronics 506 may include processing signals from the sensors in the cartridge, in one embodiment, and/or a system (such as a BCB system) that can be configured to measure dielectric changes within medium via an integrated resonant sensor, in another embodiment. In some cases, the portable sensor electronics 506 may include a detector configured to measure changes in permittivity of the resonant sensor of the sensor cartridge 504.

The distributed sensor network 508 may refer to the integration of other sensors which may be associated with and/or connected to the rapid AST 502. For example, in one embodiment, a rapid AST 502 may be proximally near a device (wireless sensor, BCB system, IoT device, etc.) that can interrogate the resonant sensor of the rapid AST 502. In such a system, the dielectric changes may be detected and passed from the rapid AST 502 to a second (or any additional) device, including any device to which the second device may be connected (either directly or via the internet).

In one embodiment, the rapid AST 502 be configured to communicate with other devices. In this manner, the rapid AST 502 may sense via the resonant sensor the dielectric changes of the medium, and communicate (via sent data packets) to another device for transmission and further processing. In such a configuration, a single rapid AST 502 vial may serve as both a sensing device (via the resonant sensor) and a transmission device (via the wireless sensor), and may be used to communicate with other rapid AST 502 devices to transmit appropriately data associated with the changes in the dielectric property of the medium.

From this perspective therefore, the rapid AST 502 represents the possibility of a smart lab or smart testing, where each vial may be equipped to sense, and/or sense and transmit data associated with dielectric changes of the medium. Such an arrangement would allow faster deployment and feedback of AST. It is to be appreciated that the wireless arrangement and distributed arrangement of the rapid AST 502 vials may be in any manner (linear, device-to-device, mesh, etc.). Further information relating to use of the rapid AST within the context of a sensors-as-a-service framework may be found in U.S. patent application Ser. No. 18/440,806, filed Feb. 13, 2024, entitled “RECONFIGURING A SECOND TYPE OF SENSOR BASED ON SENSING DATA OF A FIRST TYPE OF SENSOR,” which is hereby incorporated by reference for all purposes.

The analytics/solution integration 510 may represent components that analyze the data from the sensors and provides interpretable results. In some cases, the analytics/solution integration 510 may include a processor configured to analyze the changes in permittivity detected by the resonant sensor to determine antimicrobial susceptibility of the microorganisms. As discussed hereinabove, the transmission of data originating from the vials (e.g. sensor cartridges 504) may be subsequently processed and analyzed.

As one example, a hospital may desire to know the current real-time status of any potential sepsis case within the hospital. An analytics/solution integration 510 may be connected to a hospital lab equipped with the rapid AST 502. In one embodiment, therefore, the real-time status may include a reporting mechanism of the results of the medium test. In another embodiment, the analytics/solution integration 510 may include further processing of the data (including likely origination source, potential for additional contamination, etc.). In this manner, the hospital may not only be apprised in real-time of cases of sepsis, but also be apprised of the ramification of detected sepsis cases, as well as ways to remediate such detected cases. This ability to remediate detected cases may yield significant cost savings for hospitals, as well as significant patient quality delivery output (in terms of being able to diagnose and remedy more quickly to the patient's benefit).

In this manner, the diagram 500 shows various processing of numerous samples, real-time monitoring of the samples for bacterial growth detection, and rapid and accurate determination of antimicrobial susceptibility.

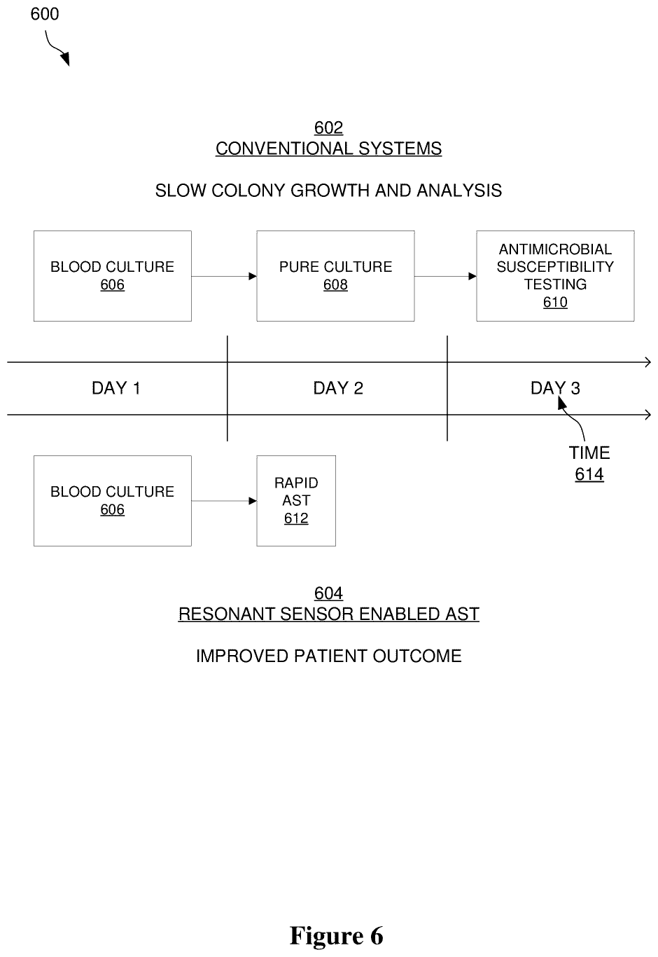

FIG. 6 depicts a comparison 600 of conventional and resonant sensor enabled antimicrobial susceptibility testing systems, according to an embodiment. As an option, the comparison 600 may be implemented in the context of any one or more of the embodiments set forth in any previous and/or subsequent figure(s) and/or description thereof. Of course, however, the comparison 600 may be implemented in the context of any desired environment. Further, the aforementioned definitions may equally apply to the description below.

As shown, the comparison 600 includes comparing conventional antimicrobial susceptibility testing (AST) systems 602 with a resonant sensor enabled AST system 604. The diagram comparison 600 emphasizes the potential for improved patient outcomes through the use of resonant sensor enabled AST.

The conventional systems 602 section shows a slow colony growth and analysis process. It begins with blood culture 606, followed by pure culture 608, and finally antimicrobial susceptibility testing 610. This process is depicted along a time axis 614, spanning three days. This conventional process involves overnight colony growth, which is inadequate when urgent determination of effective antibiotics is required, particularly for organisms resistant to primary, secondary, and tertiary antibiotics. It is recognized and acknowledged that faster AST systems exist, but the high price of using conventional faster AST systems would make system-wide use infeasible. Additionally, such faster AST systems are often more focused in what they can/cannot detect, making them impractical for the wide demands of AST needs.

On the other hand, the resonant sensor enabled AST system 604 section shows a more rapid process. It starts with blood culture 606 and proceeds directly to rapid AST 612. This streamlined process is shown to take significantly less time compared to the conventional system. In some aspects, the resonant sensor enabled AST system 604 can provide faster results compared to conventional AST systems 602. This is because the resonant sensor enabled AST system 604 leverages the use of resonant sensors to monitor bacterial growth in the antimicrobial susceptibility test panel. The resonant sensors can monitor dielectric changes in the growth media, providing an immediate reflection of the antibiotic's impact on the cellular activity of susceptible cells. This occurs much faster than the cell death relied upon by other testing methods, enabling the resonant sensor to deliver rapid and reliable results.

In some cases, the resonant sensor enabled AST system 604 may also include a processor configured to analyze the changes in permittivity detected by the resonant sensor to determine antimicrobial susceptibility of the microorganisms. The processor may also be configured to generate a report indicating antimicrobial susceptibility results for different antimicrobial agents tested in the multiple wells of the antimicrobial susceptibility testing panel.

In other aspects, the resonant sensor enabled AST system 604 may be integrated with existing AST panels, eliminating the need for additional expensive components. This innovative approach holds inherent compatibility with low-skilled environments as it has the potential to eliminate the requirement for sample preparation from positive blood cultures. This versatility makes the resonant sensor enabled AST system 604 well-suited for advanced laboratories in the developed world, where it can significantly accelerate the time-to-answer, as well as low-skill environments in the developing world.

It is recognized that the processing time of a resonant sensor enabled AST system 604 may vary. For example, in one embodiment, it may be within a day or two, or even within hours. Such a span may be dependent on the medium used and bacteria that grows. In any case, regardless of the amount of time, the emphasis of the comparison 600 is that the resonant sensor enabled AST system 604 requires a significantly less amount (usually a fraction of) the amount of time required for the conventional system 602.

As such, the resonant sensor enabled AST system 604 provides for significantly greater and improved patient outcome and care, compared to the conventional system 602 which require slow colony growth and analysis.

In various embodiments, antibiotic-resistant bacterial infections pose a significant threat in healthcare and community settings, with a growing number of bacterial species developing resistance to life-saving antibiotics. These multi-drug resistant infections have become extremely challenging to treat, as new forms of antibiotic resistance continue to emerge and spread rapidly across borders. In the United States alone, millions of people contract antibiotic-resistant infections annually, resulting in tens of thousands of deaths. The economic burden on the healthcare system is substantial, encompassing prolonged hospital stays, expensive treatment regimens, additional medical visits, and significant costs estimated to be between $20 and $35 billion, in addition to direct healthcare expenses.

To effectively combat this escalating problem, there is an increasing need for rapid, accurate, and cost-effective antibiotic susceptibility tests (AST). Traditional methods, involving overnight colony growth, are inadequate when urgent determination of effective antibiotics is required, particularly for organisms resistant to primary, secondary, and tertiary antibiotics like Carbapenem-resistant Enterobacteriaceae (CRE). The development of rapid AST methods has been hindered by limitations such as lack of multiplexing capability or significantly higher costs compared to legacy methods, impeding their widespread adoption in clinical practice.

Therefore, as disclosed herein, a crucial solution lies in the development of a rapid AST platform that can provide fully multiplexed minimum inhibitory concentration (MIC) results within hours, at a comparable cost to current methods. Such a platform would enable widespread usage, expedite the identification of effective targeted therapies, and contribute to shorter hospital stays, reduced laboratory testing, and decreased morbidity, mortality, and associated healthcare expenses. In this proposal, we present a method that fulfills these criteria, supported by preliminary data showcasing its performance and the feasibility of engineering a commercial instrument based on this method within the designated timeframe.

The presently disclosed passive resonant sensor could offer a groundbreaking solution for determining phenotypic antibiotic susceptibility with remarkable speed and affordability. By directly analyzing positive culture samples, the resonant sensor can be applied to an AST test panel. When antibiotics are introduced to a positive blood culture, the resonant sensor could detect a susceptibility-dependent and antibiotic concentration-dependent shift in its response. This shift in the sensor's dielectric sensitivity is expected to be an immediate reflection of the antibiotic's impact on the cellular activity of susceptible cells, occurring much faster than the cell death relied upon by other testing methods. This could enable the resonant sensor to deliver rapid and reliable results, providing a cost-effective approach to phenotypic antibiotic susceptibility testing.

Furthermore, the passive resonant sensor could provide inherent cost-effectiveness by utilizing an existing AST panel as the only consumable (e.g., the sensor is permanent), eliminating the need for additional expensive components. Furthermore, this innovative approach could hold inherent compatibility with low-skilled environments as it has the potential to eliminate the requirement for sample preparation from positive blood cultures. This versatility could make the passive resonant sensor well-suited for advanced laboratories in the developed world, where it could significantly accelerate the time-to-answer, as well as low-skill environments in the developing world. By combining affordability and accessibility, the resonant sensor has the power to revolutionize antibiotic susceptibility testing on a global scale.

Existing AST platforms encompass both manual and automated/semi-automated systems that assess bacterial growth in the presence of specific antibiotic concentrations to determine susceptibility. Manual methods include broth dilution tests in liquid media, as well as the antimicrobial gradient method (antibiotic gradient strips) and the disk diffusion method on solid media. These techniques necessitate overnight incubation to obtain colonies, followed by another overnight incubation to monitor growth inhibition. Consequently, it often takes at least two days from a positive blood culture to obtain actionable results.

In various embodiments, the present disclosure includes an automated AST instrument based on the resonant sensor technology disclosed herein. This instrument combines the power of the resonant sensor with the existing AST panel to monitor the effectiveness of different antibiotics at varying concentrations. By integrating this advanced sensor technology, our automated AST instrument offers a comprehensive solution for accurate and efficient antibiotic susceptibility testing.

In various embodiments, the AST Panel may include a resonant sensor that the AST system could be integrated with. Each well in the panel may contain a distinct antibiotic, and the resonant sensor may precisely measure the bacterial growth within each well over a specific time frame. This dynamic monitoring enables the detection and analysis of bacterial growth patterns when exposed to different antibiotics. By observing the changes in bacterial growth, the resonant sensor provides valuable insights into antibiotic effectiveness and susceptibility. It is to be appreciated that, as disclosed herein, the measurement of bacterial growth may occur within or outside the vial, within or outside the well (such as of a BCB system), etc. Additionally, while the application of this AST technology is focused on bacterial AST, it is to be appreciated that such a system may be applied to other pathogenic microorganism, including fungal infection.

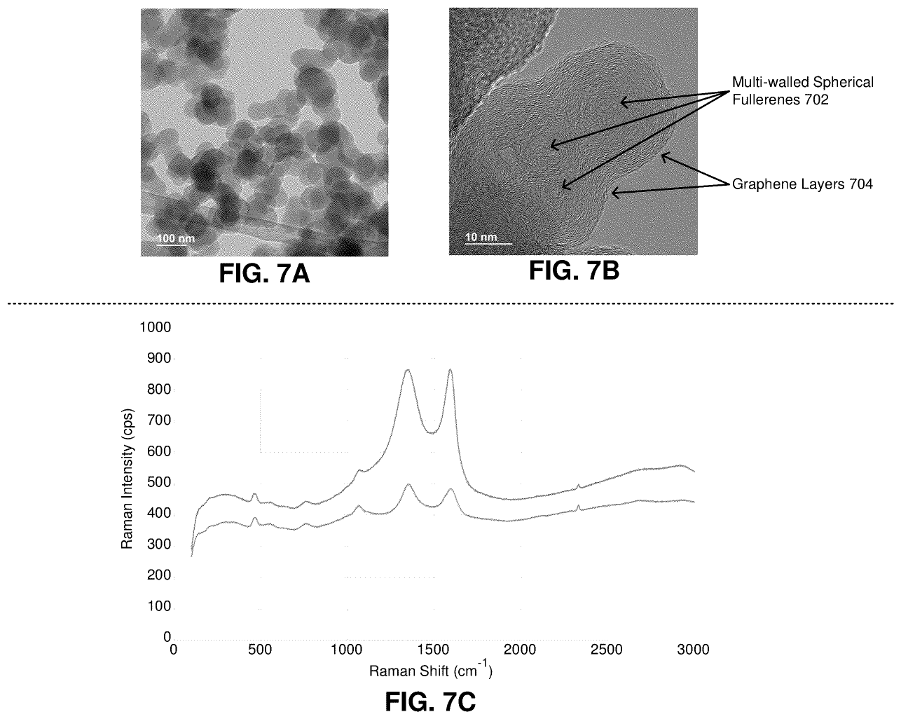

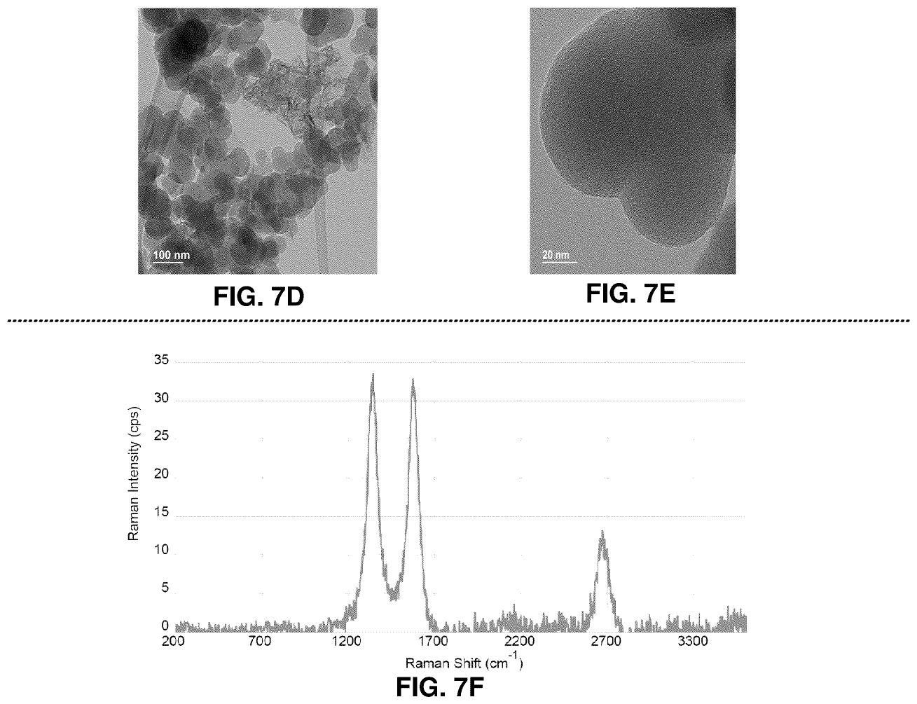

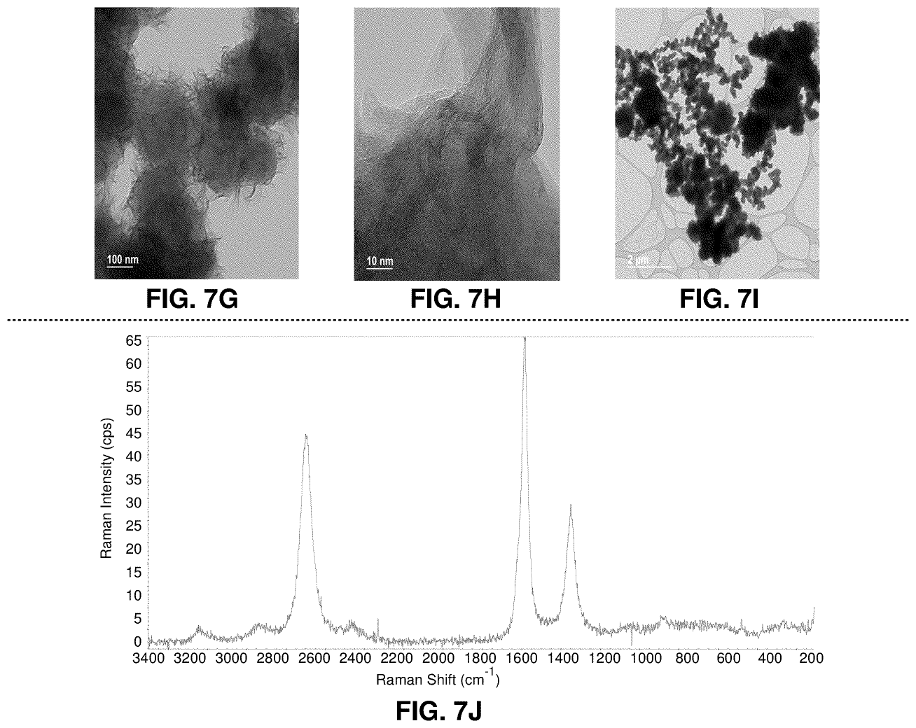

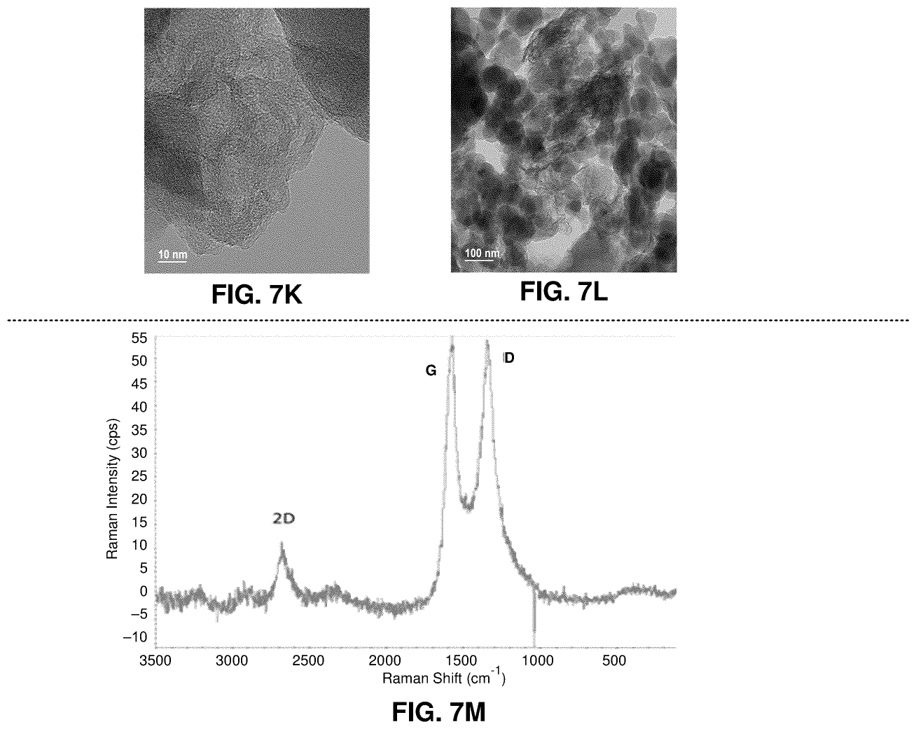

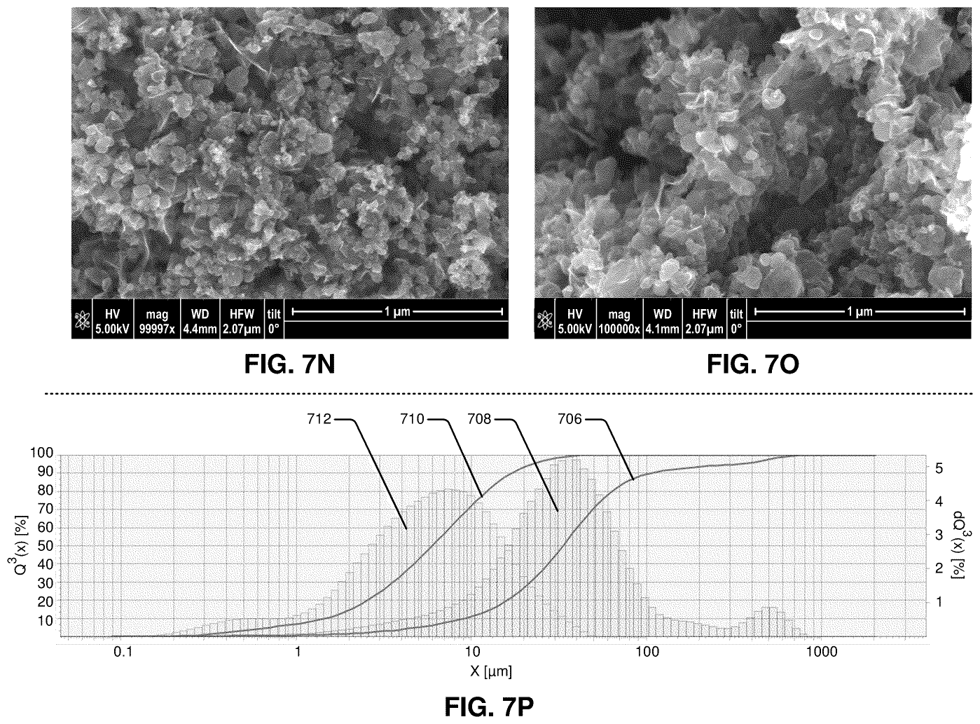

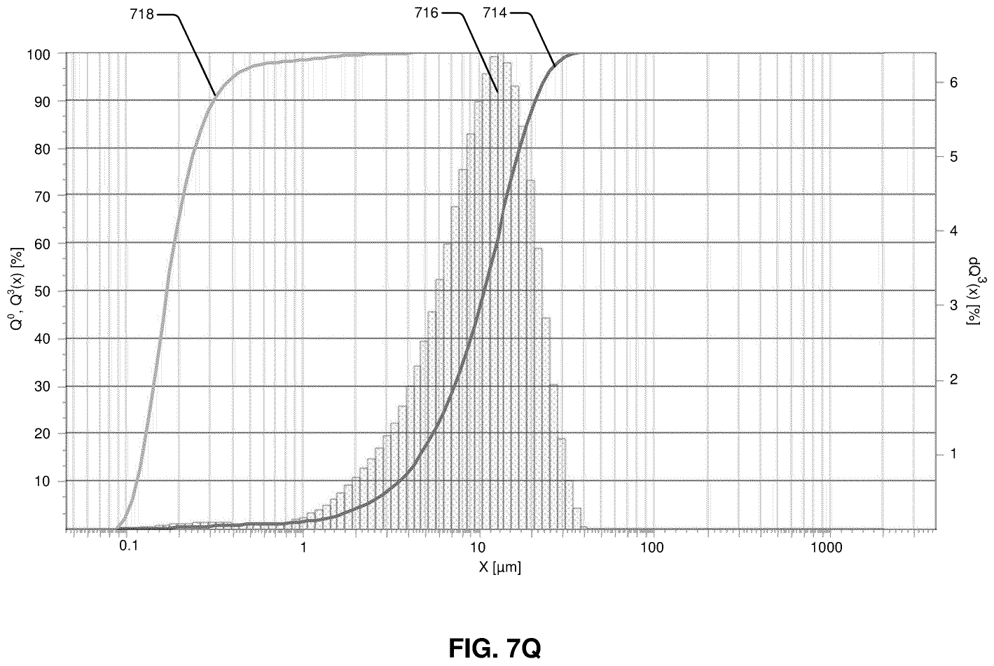

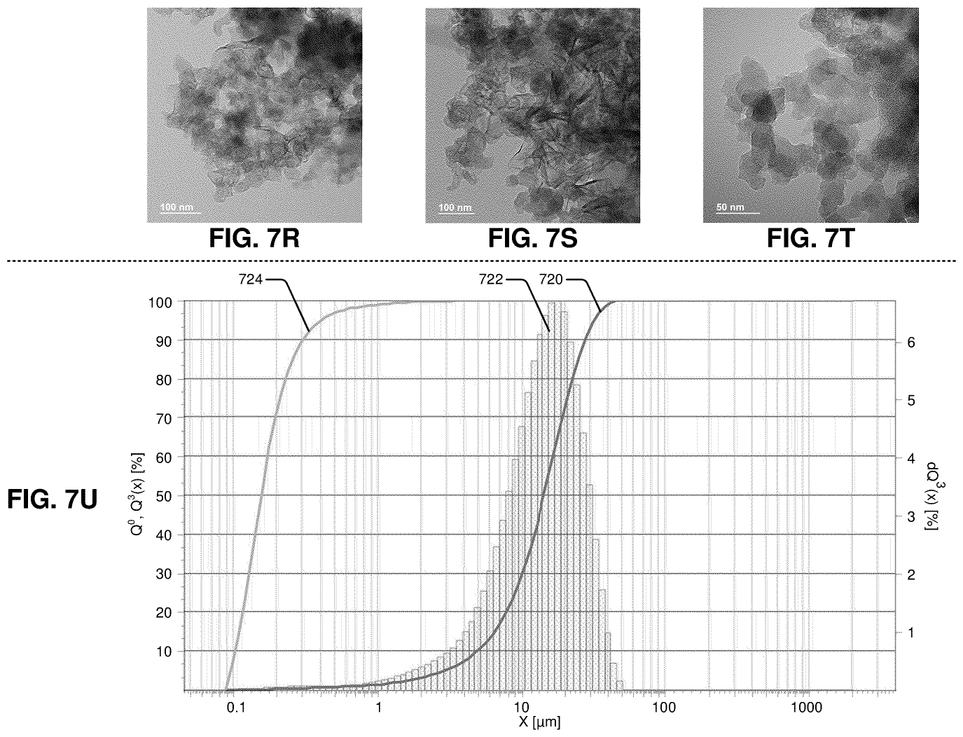

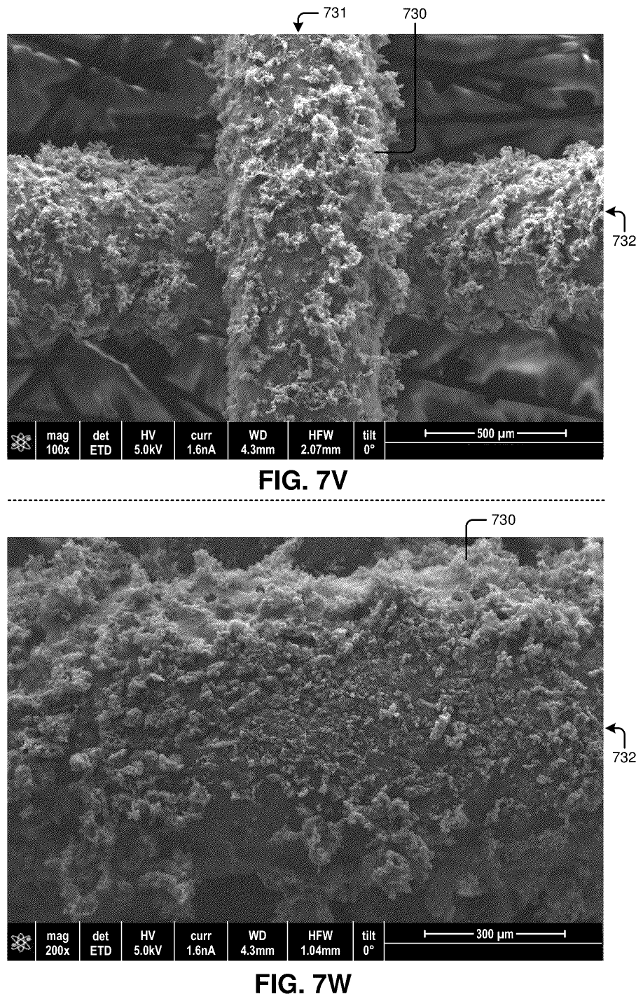

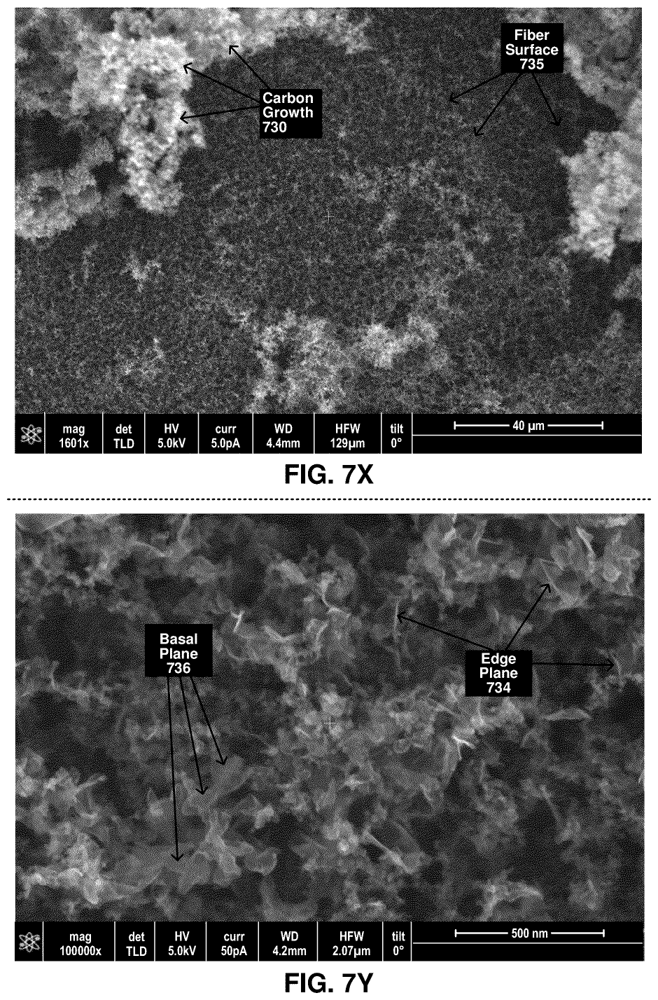

FIG. 7A through FIG. 7Y depict structured carbons, various carbon nanoparticles, various carbon-containing aggregates, and various three-dimensional carbon-containing structures that are grown over other materials, according to some embodiments of the present disclosure.

In some embodiments, the carbon nanoparticles and aggregates are characterized by a high “uniformity” (i.e., high mass fraction of desired carbon allotropes), a high degree of “order” (i.e., low concentration of defects), and/or a high degree of “purity” (i.e., low concentration of elemental impurities), in contrast to the lower uniformity, less ordered, and lower purity particles achievable with conventional systems and methods.

In some embodiments, the nanoparticles produced using the methods described herein contain multi-walled spherical fullerenes (MWSFs) or connected MWSFs and have a high uniformity (e.g., a ratio of graphene to MWSF from 20% to 80%), a high degree of order (e.g., a Raman signature with an ID/IG ratio from 0.95 to 1.05), and a high degree of purity (e.g., the ratio of carbon to other elements (other than hydrogen) is greater than 99.9%). In some embodiments, the nanoparticles produced using the methods described herein contain MWSFs or connected MWSFs, and the MWSFs do not contain a core composed of impurity elements other than carbon. In some cases, the particles produced using the methods described herein are aggregates containing the nanoparticles described above with large diameters (e.g., greater than 10 μm across).

Conventional methods have been used to produce particles containing multi-walled spherical fullerenes with a high degree of order, but the conventional methods lead to carbon products with a variety of shortcomings. For example, high temperature synthesis techniques lead to particles with a mixture of many carbon allotropes and therefore low uniformity (e.g., less than 20% fullerenes to other carbon allotropes) and/or small particle sizes (e.g., less than lum, or less than 100 nm in some cases). Methods using catalysts lead to products including the catalyst elements and therefore have low purity (e.g., less than 95% carbon to other elements) as well. These undesirable properties also often lead to undesirable electrical properties of the resulting carbon particles (e.g., electrical conductivity of less than 1000 S/m).

In some embodiments, the carbon nanoparticles and aggregates described herein are characterized by Raman spectroscopy that is indicative of the high degree of order and uniformity of structure. In some embodiments, the uniform, ordered and/or pure carbon nanoparticles and aggregates described herein are produced using relatively high speed, low cost improved thermal reactors and methods, as described below. Additional advantages and/or improvements will also become apparent from the following disclosure.

In the present disclosure, the term “graphene” refers to an allotrope of carbon in the form of a two-dimensional, atomic-scale, hexagonal lattice in which one atom forms each vertex. The carbon atoms in graphene are sp2 bonded. Additionally, graphene has a Raman spectrum with two main peaks: a G-mode at approximately 1580 cm-1 and a D mode at approximately 1350 cm-1 (when using a 532 nm excitation laser).

In the present disclosure, the term “fullerene” refers to a molecule of carbon in the form of a hollow sphere, ellipsoid, tube, or other shapes. Spherical fullerenes can also be referred to as Buckminsterfullerenes, or buckyballs. Cylindrical fullerenes can also be referred to as carbon nanotubes. Fullerenes are similar in structure to graphite, which is composed of stacked graphene sheets of linked hexagonal rings. Fullerenes may also contain pentagonal (or sometimes heptagonal) rings.

In the present disclosure, the term “multi-walled fullerene” refers to fullerenes with multiple concentric layers. For example, multi-walled nanotubes (MWNTs) contain multiple rolled layers (concentric tubes) of graphene. Multi-walled spherical fullerenes (MWSFs) contain multiple concentric spheres of fullerenes.

In the present disclosure, the term “nanoparticle” refers to a particle that measures from 1 nm to 989 nm. The nanoparticle can include one or more structural characteristics (e.g., crystal structure, defect concentration, etc.), and one or more types of atoms. The nanoparticle can be any shape, including but not limited to spherical shapes, spheroidal shapes, dumbbell shapes, cylindrical shapes, elongated cylindrical type shapes, rectangular prism shapes, disk shapes, wire shapes, irregular shapes, dense shapes (i.e., with few voids), porous shapes (i.e., with many voids), etc.

In the present disclosure, the term “aggregate” refers to a plurality of nanoparticles that are connected together by electrostatic forces (e.g., Van der Waals forces, London dispersion forces, dipole-dipole interactions, hydrogen bonding, etc.) by covalent bonds, by ionic bonds, by metallic bonds, or by other physical or chemical interactions. Aggregates can vary in size considerably, but in general are larger than about 500 nm.

In some embodiments, a carbon nanoparticle, as described herein, includes two or more connected multi-walled spherical fullerenes (MWSFs) and layers of graphene coating the connected MWSFs. In some embodiments, a carbon nanoparticle, as described herein, includes two or more connected multi-walled spherical fullerenes (MWSFs) and layers of graphene coating the connected MWSFs where the MWSFs do not contain a core composed of impurity elements other than carbon. In some embodiments, a carbon nanoparticle, as described herein, includes two or more connected multi-walled spherical fullerenes (MWSFs) and layers of graphene coating the connected MWSFs where the MWSFs do not contain a void (i.e., a space with no carbon atoms greater than approximately 0.5 nm, or greater than approximately 1 nm) at the center. In some embodiments, the connected MWSFs are formed of concentric, well-ordered spheres of sp2-hybridized carbon atoms, as contrasted with spheres of poorly-ordered, non-uniform, amorphous carbon particles.

In some embodiments, the nanoparticles containing the connected MWSFs have an average diameter in a range from 5 to 500 nm, or from 5 to 250 nm, or from 5 to 100 nm, or from 5 to 50 nm, or from 10 to 500 nm, or from 10 to 250 nm, or from 10 to 100 nm, or from 10 to 50 nm, or from 40 to 500 nm, or from 40 to 250 nm, or from 40 to 100 nm, or from 50 to 500 nm, or from 50 to 250 nm, or from 50 to 100 nm. Of course, nanoparticles containing connected MWSFs may have an average diameter characterized by having any of the foregoing values or being within any of the foregoing exemplary ranges, or an average diameter characterized by having a value or being within a range between any of the foregoing exemplary ranges, without limitation and without departing from the scope of the presently described inventive concepts.

In some embodiments, the carbon nanoparticles described herein form aggregates, wherein many nanoparticles aggregate together to form a larger unit. In some embodiments, a carbon aggregate includes a plurality of carbon nanoparticles. A diameter across the carbon aggregate is in a range from 10 to 500 μm, or from 50 to 500 μm, or from 100 to 500 μm, or from 250 to 500 μm, or from 10 to 250 μm, or from 10 to 100 μm, or from 10 to 50 μm. Of course, carbon aggregates may have an average diameter characterized by having any of the foregoing values or being within any of the foregoing exemplary ranges, or an average diameter characterized by having a value or being within a range between any of the foregoing exemplary ranges, without limitation and without departing from the scope of the presently described inventive concepts.

In some embodiments, the aggregate is formed from a plurality of carbon nanoparticles, as defined above. In some embodiments, aggregates contain connected MWSFs. In some embodiments, the aggregates contain connected MWSFs with a high uniformity metric (e.g., a ratio of graphene to MWSF from 20% to 80%), a high degree of order (e.g., a Raman signature with an ID/IG ratio from 0.95 to 1.05), and a high degree of purity (e.g., greater than 99.9% carbon).

One benefit of producing aggregates of carbon nanoparticles, particularly with diameters in the ranges described above, is that aggregates of particles greater than 10 μm are casier to collect than particles or aggregates of particles that are smaller than 500 nm. The case of collection reduces the cost of manufacturing equipment used in the production of the carbon nanoparticles and increases the yield of the carbon nanoparticles. Additionally, particles greater than 10 μm in size pose fewer safety concerns compared to the risks of handling smaller nanoparticles, e.g., potential health and safety risks due to inhalation of the smaller nanoparticles. The lower health and safety risks, thus, further reduce the manufacturing cost.

In some embodiments, a carbon nanoparticle has a ratio of graphene to MWSFs from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. In some embodiments, a carbon aggregate has a ratio of graphene to MWSFs is from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. Of course, carbon nanoparticles may have a graphene-to-MWSF ratio characterized by having any of the foregoing values or being within any of the foregoing exemplary ranges, or an average graphene-to-MWSF ratio characterized by having a value or being within a range between any of the foregoing exemplary ranges, without limitation and without departing from the scope of the presently described inventive concepts.

In some embodiments, a carbon nanoparticle has a ratio of graphene to connected MWSFs from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. In some embodiments, a carbon aggregate has a ratio of graphene to connected MWSFs is from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. Of course, carbon nanoparticles may have a graphene-to-connected MWSF ratio characterized by having any of the foregoing values or being within any of the foregoing exemplary ranges, or an average graphene-to-connected MWSF ratio characterized by having a value or being within a range between any of the foregoing exemplary ranges, without limitation and without departing from the scope of the presently described inventive concepts.

In some embodiments, Raman spectroscopy is used to characterize carbon allotropes to distinguish their molecular structures. For example, graphene can be characterized using Raman spectroscopy to determine information such as order/disorder, edge and grain boundaries, thickness, number of layers, doping, strain, and thermal conductivity. MWSFs have also been characterized using Raman spectroscopy to determine the degree of order of the MWSFs.

In some embodiments, Raman spectroscopy is used to characterize the structure of MWSFs or connected MWSFs. The main peaks in the Raman spectra are the G mode and the D mode. The G mode is attributed to the vibration of carbon atoms in sp2 hybridized carbon networks, and the D mode is related to the breathing of hexagonal carbon rings with defects. In some cases, defects may be present, yet may not be detectable in the Raman spectra. For example, if the presented crystalline structure is orthogonal with respect to the basal plane, the D peak will show an increase. On the other hand, if presented with a perfectly planar surface that is parallel with respect to the basal plane, the D peak will be zero.

When using 532 nm incident light, the Raman G mode is typically at 1582 cm-1 for planar graphite, however can be downshifted for MWSFs or connected MWSFs (e.g., down to 1565 cm-1 or down to1580 cm-1). The D mode is observed at approximately 1350 cm-1 in the Raman spectra of MWSFs or connected MWSFs. The ratio of the intensities of the D mode peak to G mode peak (i.e., the ID/IG) is related to the degree of order of the MWSFs, where a lower ID/IG indicates a higher degree of order. An ID/IG near or below 1 indicates a relatively high degree of order, and an ID/IG greater than 1.1 indicates a lower degree of order.

In some embodiments, a carbon nanoparticle or a carbon aggregate containing MWSFs or connected MWSFs, as described herein, has a Raman spectrum with a first Raman peak at about 1350 cm 1 and a second Raman peak at about 1580 cm 1 when using 532 nm incident light. In some embodiments, the ratio of an intensity of the first Raman peak to an intensity of the second Raman peak (i.e., the ID/IG) for the nanoparticles or the aggregates described herein is in a range from 0.95 to 1.05, or from 0.9 to 1.1, or from 0.8 to 1.2, or from 0.9 to 1.2, or from 0.8 to 1.1, or from 0.5 to 1.5, or less than 1.5, or less than 1.2, or less than 1.1, or less than 1, or less than 0.95, or less than 0.9, or less than 0.8. Of course, carbon nanoparticles or aggregates including MWSFs or connected MWSFs may be characterized by a ratio of first and second Raman peak intensities having any of the foregoing values or being within any of the foregoing exemplary ranges, or a ratio of first and second Raman peak intensities characterized by having a value or being within a range between any of the foregoing exemplary ranges, without limitation and without departing from the scope of the presently described inventive concepts.

In some embodiments, a carbon aggregate containing MWSFs or connected MWSFs, as defined above, has a high purity. In some embodiments, the carbon aggregate containing MWSFs or connected MWSFs has a ratio of carbon to metals of greater than 99.99%, or greater than 99.95%, or greater than 99.9%, or greater than 99.8%, or greater than 99.5%, or greater than 99%. In some embodiments, the carbon aggregate has a ratio of carbon to other elements of greater than 99.99%, or greater than 99.95%, or greater than 99.9%, or greater than 99.5%, or greater than 99%, or greater than 90%, or greater than 80%, or greater than 70%, or greater than 60%. In some embodiments, the carbon aggregate has a ratio of carbon to other elements (except for hydrogen) of greater than 99.99%, or greater than 99.95%, or greater than 99.9%, or greater than 99.8%, or greater than 99.5%, or greater than 99%, or greater than 90%, or greater than 80%, or greater than 70%, or greater than 60%. Of course, carbon aggregates including MWSFs or connected MWSFs may be characterized by a ratio of carbon to metal having any of the foregoing values or being within any of the foregoing exemplary ranges, or a ratio of carbon to metal having value or being within a range between any of the foregoing exemplary ranges, without limitation and without departing from the scope of the presently described inventive concepts.

In some embodiments, a carbon aggregate containing MWSFs or connected MWSFs, as defined above, has a high specific surface area. In some embodiments, the carbon aggregate has a Brunauer, Emmett and Teller (BET) specific surface area from 10 to 200 m2/g, or from 10 to 100 m2/g, or from 10 to 50 m2/g, or from 50 to 200 m2/g, or from 50 to 100 m2/g, or from 10 to 1000 m2/g. Of course, carbon aggregates including MWSFs or connected MWSFs may be characterized by a BET specific surface area having any of the foregoing values or being within any of the foregoing exemplary ranges, or a BET specific surface area characterized by having a value or being within a range between any of the foregoing exemplary ranges, without limitation and without departing from the scope of the presently described inventive concepts.

In some embodiments, a carbon aggregate containing MWSFs or connected MWSFs, as defined above, has a high electrical conductivity. In some embodiments, a carbon aggregate containing MWSFs or connected MWSFs, as defined above, is compressed into a pellet and the pellet has an electrical conductivity greater than 500 S/m, or greater than 1000 S/m, or greater than 2000 S/m, or greater than 3000 S/m, or greater than 4000 S/m, or greater than 5000 S/m, or greater than 10000 S/m, or greater than 20000 S/m, or greater than 30000 S/m, or greater than 40000 S/m, or greater than 50000 S/m, or greater than 60000 S/m, or greater than 70000 S/m, or from 500 S/m to 100000 S/m, or from 500 S/m to 1000 S/m, or from 500 S/m to 10000 S/m, or from 500 S/m to 20000 S/m, or from 500 S/m to 100000 S/m, or from 1000 S/m to 10000 S/m, or from 1000 S/m to 20000 S/m, or from 10000 to 100000 S/m, or from 10000 S/m to 80000 S/m, or from 500 S/m to 10000 S/m. Of course, carbon aggregates including MWSFs or connected MWSFs may be characterized by an electrical conductivity having any of the foregoing values or being within any of the foregoing exemplary ranges, or an electrical conductivity characterized by having a value or being within a range between any of the foregoing exemplary ranges, without limitation and without departing from the scope of the presently described inventive concepts.

In some cases, the density of the pellet is approximately 1 g/cm³, or approximately 1.2 g/cm³, or approximately 1.5 g/cm³, or approximately 2 g/cm³, or approximately 2.2 g/cm³, or approximately 2.5 g/cm³, or approximately 3 g/cm³. Of course, pellets may be characterized by a density having any of the foregoing values or being within any of the foregoing exemplary ranges, or a density having a value or being within a range between any of the foregoing exemplary ranges, without limitation and without departing from the scope of the presently described inventive concepts.

Additionally, tests have been performed in which compressed pellets of the carbon aggregate materials have been formed with compressions of 2000 psi and 12000 psi and with annealing temperatures of 800° C. and 1000° C. The higher compression and/or the higher annealing temperatures generally result in pellets with a higher degree of electrical conductivity, including in the range of 12410.0 S/m to 13173.3 S/m.

### High Purity Carbon Allotropes Produced Using Thermal Processing Systems

In some embodiments, the carbon nanoparticles and aggregates described herein are produced using thermal reactors and methods, such as any appropriate thermal reactor and/or method. Further details pertaining to thermal reactors and/or methods of use can be found in U.S. Pat. No. 9,862,602, issued Jan. 9, 2018, titled “CRACKING OF A PROCESS GAS”, which is hereby incorporated by reference in its entirety. Additionally, precursors (e.g., including methane, ethane, propane, butane, and natural gas) can be used with the thermal reactors to produce the carbon nanoparticles and the carbon aggregates described herein.

In some embodiments, the carbon nanoparticles and aggregates described herein are produced using the thermal reactors with gas flow rates from 1 slm to 10 slm, or from 0.1 slm to 20 slm, or from 1 slm to 5 slm, or from 5 slm to 10 slm, or greater than 1 slm, or greater than 5 slm. In some embodiments, the carbon nanoparticles and aggregates described herein are produced using the thermal reactors with gas resonance times from 0.1 seconds to 30 seconds, or from 0.1 seconds to 10 seconds, or from 1 seconds to 10 seconds, or from 1 seconds to 5 seconds, from 5 seconds to 10 seconds, or greater than 0.1 seconds, or greater than 1 seconds, or greater than 5 seconds, or less than 30 seconds. Of course, carbon nanoparticles and aggregates may be produced using thermal reactors with gas flow rates having any of the foregoing values or being within any of the foregoing exemplary ranges, or gas flow rates having a value or being within a range between any of the foregoing exemplary ranges, without limitation and without departing from the scope of the presently described inventive concepts.

In some embodiments, the carbon nanoparticles and aggregates described herein are produced using the thermal reactors with production rates from 10 g/hr to 200 g/hr, or from 30 g/hr to 200 g/hr, or from 30 g/hr to 100 g/hr, or from 30 g/hr to 60 g/hr, or from 10 g/hr to 100 g/hr, or greater than 10 g/hr, or greater than 30 g/hr, or greater than 100 g/hr. Of course, carbon nanoparticles and aggregates may be produced using thermal reactors with production rates having any of the foregoing values or being within any of the foregoing exemplary ranges, or production rates having a value or being within a range between any of the foregoing exemplary ranges, without limitation and without departing from the scope of the presently described inventive concepts.

In some embodiments, thermal reactors or other cracking apparatuses and thermal reactor methods or other cracking methods can be used for refining, pyrolizing, dissociating or cracking feedstock process gases into its constituents to produce the carbon nanoparticles and the carbon aggregates described herein, as well as other solid and/or gaseous products (e.g., hydrogen gas and/or lower order hydrocarbon gases). The feedstock process gases generally include, for example, hydrogen gas (H2), carbon dioxide (CO2), C1 to C10 hydrocarbons, aromatic hydrocarbons, and/or other hydrocarbon gases such as natural gas, methane, ethane, propane, butane, isobutane, saturated/unsaturated hydrocarbon gases, ethene, propene, etc., and mixtures thereof. The carbon nanoparticles and the carbon aggregates can include, for example, multi-walled spherical fullerenes (MWSFs), connected MWSFs, carbon nanospheres, graphene, graphite, highly ordered pyrolytic graphite, single-walled nanotubes, multi-walled nanotubes, other solid carbon products, and/or the carbon nanoparticles and the carbon aggregates described herein.

Some embodiments for producing the carbon nanoparticles and the carbon aggregates described herein include thermal cracking methods that use, for example, an elongated longitudinal heating element optionally enclosed within an elongated casing, housing or body of a thermal cracking apparatus. The body generally includes, for example, one or more tubes or other appropriate enclosures made of stainless steel, titanium, graphite, quartz, or the like. In some embodiments, the body of the thermal cracking apparatus is generally cylindrical in shape with a central elongate longitudinal axis arranged vertically and a feedstock process gas inlet at or near a top of the body. The feedstock process gas flows longitudinally down through the body or a portion thereof. In the vertical configuration, both gas flow and gravity assist in the removal of the solid products from the body of the thermal cracking apparatus.

The heating element generally includes, for example, a heating lamp, one or more resistive wires or filaments (or twisted wires), metal filaments, metallic strips or rods, and/or other appropriate thermal radical generators or elements that can be heated to a specific temperature (i.e., a molecular cracking temperature) sufficient to thermally crack molecules of the feedstock process gas. The heating element is generally disposed, located or arranged to extend centrally within the body of the thermal cracking apparatus along the central longitudinal axis thereof. For example, if there is only one heating element, then it is placed at or concentric with the central longitudinal axis, and if there is a plurality of the heating elements, then they are spaced or offset generally symmetrically or concentrically at locations near and around and parallel to the central longitudinal axis.

Thermal cracking to produce the carbon nanoparticles and aggregates described herein is generally achieved by passing the feedstock process gas over, or in contact with, or within the vicinity of, the heating element within a longitudinal elongated reaction zone generated by heat from the heating element and defined by and contained inside the body of the thermal cracking apparatus to heat the feedstock process gas to or at a specific molecular cracking temperature.

The reaction zone is considered to be the region surrounding the heating element and close enough to the heating element for the feedstock process gas to receive sufficient heat to thermally crack the molecules thereof. The reaction zone is thus generally axially aligned or concentric with the central longitudinal axis of the body. In some embodiments, the thermal cracking is performed under a specific pressure. In some embodiments, the feedstock process gas is circulated around or across the outside surface of a container of the reaction zone or a heating chamber in order to cool the container or chamber and preheat the feedstock process gas before flowing the feedstock process gas into the reaction zone.

In some embodiments, the carbon nanoparticles and aggregates described herein and/or hydrogen gas are produced without the use of catalysts. In other words, the process is catalyst free.

Some embodiments to produce the carbon nanoparticles and aggregates described herein using thermal cracking apparatuses and methods to provide a standalone system that can advantageously be rapidly scaled up or scaled down for different production levels as desired. For example, some embodiments are scalable to provide a standalone hydrogen and/or carbon nanoparticle producing station, a hydrocarbon source, or a fuel cell station. Some embodiments can be scaled up to provide higher capacity systems, e.g., for a refinery or the like.

In some embodiments, a thermal cracking apparatus for cracking a feedstock process gas to produce the carbon nanoparticles and aggregates described herein include a body, a feedstock process gas inlet, and an elongated heating element. The body has an inner volume with a longitudinal axis. The inner volume has a reaction zone concentric with the longitudinal axis. A feedstock process gas is flowed into the inner volume through the feedstock process gas inlet during thermal cracking operations. The elongated heating element is disposed within the inner volume along the longitudinal axis and is surrounded by the reaction zone. During the thermal cracking operations, the elongated heating element is heated by electrical power to a molecular cracking temperature to generate the reaction zone, the feedstock process gas is heated by heat from the elongated heating element, and the heat thermally cracks molecules of the feedstock process gas that are within the reaction zone into constituents of the molecules.

In some embodiments, a method for cracking a feedstock process gas to produce the carbon nanoparticles and aggregates described herein includes: (1) providing a thermal cracking apparatus having an inner volume that has a longitudinal axis and an elongated heating element disposed within the inner volume along the longitudinal axis; (2) heating the elongated heating element by electrical power to a molecular cracking temperature to generate a longitudinal elongated reaction zone within the inner volume; (3) flowing a feedstock process gas into the inner volume and through the longitudinal elongated reaction zone (e.g., wherein the feedstock process gas is heated by heat from the elongated heating element); and (4) thermally cracking molecules of the feedstock process gas within the longitudinal elongated reaction zone into constituents thereof (e.g., hydrogen gas and one or more solid products) as the feedstock process gas flows through the longitudinal elongated reaction zone.

In some embodiments, the feedstock process gas to produce the carbon nanoparticles and aggregates described herein includes a hydrocarbon gas. The results of cracking include hydrogen (e.g., H2) and various forms of the carbon nanoparticles and aggregates described herein. In some embodiments, the carbon nanoparticles and aggregates include two or more MWSFs and layers of graphene coating the MWSFs, and/or connected MWSFs and layers of graphene coating the connected MWSFs. In some embodiments, the feedstock process gas is preheated (e.g., to 100° C. to 500° C.) by flowing the feedstock process gas through a gas preheating region between a heating chamber and a shell of the thermal cracking apparatus before flowing the feedstock process gas into the inner volume. In some embodiments, a gas having nanoparticles therein is flowed into the inner volume and through the longitudinal elongated reaction zone to mix with the feedstock process gas, and a coating of a solid product (e.g., layers of graphene) is formed around the nanoparticles.

### Post-Processing High Purity Structured Carbons

In some embodiments, the carbon nanoparticles and aggregates containing multi-walled spherical fullerenes (MWSFs) or connected MWSFs described herein are produced and collected, and no post-processing is done. In other embodiments, the carbon nanoparticles and aggregates containing multi-walled spherical fullerenes (MWSFs) or connected MWSFs described herein are produced and collected, and some post-processing is done. Some examples of post-processing include mechanical processing such as ball milling, grinding, attrition milling, micro fluidizing, and other techniques to reduce the particle size without damaging the MWSFs. Some further examples of post-processing include exfoliation processes such as sheer mixing, chemical etching, oxidizing (e.g., Hummer method), thermal annealing, doping by adding elements during annealing (e.g., sulfur, nitrogen), steaming, filtering, and lyophilizing, among others. Some examples of post-processing include sintering processes such as spark plasma sintering (SPS), direct current sintering, microwave sintering, and ultraviolet (UV) sintering, which can be conducted at high pressure and temperature in an inert gas. In some embodiments, multiple post-processing methods can be used together or in a series. In some embodiments, the post-processing produces functionalized carbon nanoparticles or aggregates containing multi-walled spherical fullerenes (MWSFs) or connected MWSFs.

In some embodiments, the materials are mixed together in different combinations. In some embodiments, different carbon nanoparticles and aggregates containing MWSFs or connected MWSFs described herein are mixed together before post-processing. For example, different carbon nanoparticles and aggregates containing MWSFs or connected MWSFs with different properties (e.g., different sizes, different compositions, different purities, from different processing runs, etc.) can be mixed together. In some embodiments, the carbon nanoparticles and aggregates containing MWSFs or connected MWSFs described herein can be mixed with graphene to change the ratio of the connected MWSFs to graphene in the mixture. In some embodiments, different carbon nanoparticles and aggregates containing MWSFs or connected MWSFs described herein can be mixed together after post-processing. For example, different carbon nanoparticles and aggregates containing MWSFs or connected MWSFs with different properties and/or different post-processing methods (e.g., different sizes, different compositions, different functionality, different surface properties, different surface areas) can be mixed together.

In some embodiments, the carbon nanoparticles and aggregates described herein are produced and collected, and subsequently processed by mechanical grinding, milling, and/or exfoliating. In some embodiments, the processing (e.g., by mechanical grinding, milling, exfoliating, etc.) reduces the average size of the particles. In some embodiments, the processing (e.g., by mechanical grinding, milling, exfoliating, etc.) increases the average surface area of the particles. In some embodiments, the processing by mechanical grinding, milling and/or exfoliation shears off some fraction of the carbon layers, producing sheets of graphite mixed with the carbon nanoparticles.

In some embodiments, the mechanical grinding or milling is performed using a ball mill, a planetary mill, a rod mill, a shear mixer, a high-shear granulator, an autogenous mill, or other types of machining used to break solid materials into smaller pieces by grinding, crushing or cutting. In some embodiments, the mechanical grinding, milling and/or exfoliating is performed wet or dry. In some embodiments, the mechanical grinding is performed by grinding for some period of time, then idling for some period of time, and repeating the grinding and idling for a number of cycles. In some embodiments, the grinding period is from 1 minute to 20 minutes, or from 1 minute to 10 minutes, or from 3 minutes to 8 minutes, or approximately 3 minutes, or approximately 8 minutes. In some embodiments, the idling period is from 1 minute to 10 minutes, or approximately 5 minutes, or approximately 6 minutes. In some embodiments, the number of grinding and idling cycles is from 1 minute to 100 minutes, or from 5 minutes to 100 minutes, or from 10 minutes to 100 minutes, or from 5 minutes to 10 minutes, or from 5 minutes to 20 minutes. In some embodiments, the total amount of time of grinding and idling is from 10 minutes to 1200 minutes, or from 10 minutes to 600 minutes, or from 10 minutes to 240 minutes, or from 10 minutes to 120 minutes, or from 100 minutes to 90 minutes, or from 10 minutes to 60 minutes, or approximately 90 minutes, or approximately 120 minutes. Of course, grinding, milling, or idling times within the scope of the presently disclosed inventive embodiments may have any of the foregoing values or be within any of the foregoing exemplary ranges, between any of the foregoing exemplary ranges, without limitation and without departing from the scope of the presently described inventive concepts.

In some embodiments, the grinding steps in the cycle are performed by rotating a mill in one direction for a first cycle (e.g., clockwise), and then rotating a mill in the opposite direction (e.g., counterclockwise) for the next cycle. In some embodiments, the mechanical grinding or milling is performed using a ball mill, and the grinding steps are performed using a rotation speed from 100 to 1000 rpm, or from 100 to 500 rpm, or approximately 400 rpm, or any value or range of values therebetween. In some embodiments, the mechanical grinding or milling is performed using a ball mill that uses a milling media with a diameter from 0.1 mm to 20 mm, or from 0.1 mm to 10 mm, or from 1 mm to 10 mm, or approximately 0.1 mm, or approximately 1 mm, or approximately 10 mm, or any value or range of values therebetween. In some embodiments, the mechanical grinding or milling is performed using a ball mill that uses a milling media composed of metal such as steel, an oxide such as zirconium oxide (zirconia), yttria stabilized zirconium oxide, silica, alumina, magnesium oxide, or other hard materials such as silicon carbide or tungsten carbide.

In some embodiments, the carbon nanoparticles and aggregates described herein are produced and collected, and subsequently processed using elevated temperatures such as thermal annealing or sintering. In some embodiments, the processing using elevated temperatures is done in an inert environment such as nitrogen or argon. In some embodiments, the processing using elevated temperatures is done at atmospheric pressure, or under vacuum, or at low pressure. In some embodiments, the processing using elevated temperatures is done at a temperature from 500° C. to 2500° C., or from 500° C. to 1500° C., or from 800° C. to 1500° C., or from 800° C. to 1200° C., or from 800° C. to 1000° C., or from 2000° C. to 2400° C., or approximately 800° C., or approximately 1000° C., or approximately 1500° C., or approximately 2000° C., or approximately 2400° C. Of course, processing using elevated temperatures may be performed at any of the foregoing temperatures, or at a temperature within any of the foregoing exemplary ranges, or between any of the foregoing exemplary ranges, without limitation and without departing from the scope of the presently described inventive concepts.

In some embodiments, the carbon nanoparticles and aggregates described herein are produced and collected, and subsequently, in post processing steps, additional elements or compounds are added to the carbon nanoparticles, thereby incorporating the unique properties of the carbon nanoparticles and aggregates into other mixtures of materials.

In some embodiments, either before or after post-processing, the carbon nanoparticles and aggregates described herein are added to solids, liquids or slurries of other elements or compounds to form additional mixtures of materials incorporating the unique properties of the carbon nanoparticles and aggregates. In some embodiments, the carbon nanoparticles and aggregates described herein are mixed with other solid particles, polymers or other materials.

In some embodiments, either before or after post-processing, the carbon nanoparticles and aggregates described herein are used in various applications beyond applications pertaining to the present disclosure. Such applications including but not limited to transportation applications (e.g., automobile and truck tires, couplings, mounts, elastomeric o rings, hoses, sealants, grommets, etc.) and industrial applications (e.g., rubber additives, functionalized additives for polymeric materials, additives for epoxies, etc.).

FIGS. 7A and 7B show transmission electron microscope (TEM) images of as synthesized carbon nanoparticles. The carbon nanoparticles of FIG. 7A (at a first magnification) and FIG. 7B (at a second magnification) contain connected multi-walled spherical fullerenes 702 (MWSFs) with graphene layers 704 that coat the connected MWSFs. The ratio of MWSF to graphene allotropes in this example is approximately 80% due to the relatively short resonance times. The MWSFs in FIG. 7A are approximately 5 nm to 10 nm in diameter, and the diameter can be from 5 nm to 500 nm using the conditions described above. In some embodiments, the average diameter across the MWSFs is in a range from 5 nm to 500 nm, or from 5 nm to 250 nm, or from 5 nm to 100 nm, or from 5 nm to 50 nm, or from 10 nm to 500 nm, or from 10 nm to 250 nm, or from 10 nm to 100 nm, or from 10 nm to 50 nm, or from 40 nm to 500 nm, or from 40 nm to 250 nm, or from 40 nm to 100 nm, or from 50 nm to 500 nm, or from 50 nm to 250 nm, or from 50 nm to 100 nm. Of course, average MWSF diameter within the scope of the presently disclosed inventive embodiments may have any of the foregoing values or be within any of the foregoing exemplary ranges, or between any of the foregoing exemplary ranges, without limitation and without departing from the scope of the presently described inventive concepts. No catalyst was used in this process, and therefore, there is no central seed containing contaminants. The aggregate particles produced in this example had a particle size of approximately 10 μm to 100 μm, or approximately 10 μm to 500 μm.

FIG. 7C shows the Raman spectrum of the as synthesized aggregates in this example taken with 532 nm incident light. The ID/IG for the aggregates produced in this example is from approximately 0.99 to 1.03, indicating that the aggregates were composed of carbon allotropes with a high degree of order.

FIG. 7D and FIG. 7E show example TEM images of the carbon nanoparticles after size reduction by grinding in a ball mill. The ball milling was performed in cycles with a 3 minute counter-clockwise grinding step, followed by a 6 minute idle step, followed by a 3 minute clockwise grinding step, followed by a 6 minute idle step. The grinding steps were performed using a rotation speed of 400 rpm. The milling media was zirconia and ranged in size from 0.1 mm to 10 mm. The total size reduction processing time was from 60 minutes to 120 minutes. After size reduction, the aggregate particles produced in this example had a particle size of approximately 1 μm to 5 μm. The carbon nanoparticles after size reduction are connected MWSFs with layers of graphene coating the connected MWSFs.

FIG. 7F shows a Raman spectrum from these aggregates after size reduction taken with a 532 nm incident light. The ID/IG for the aggregate particles in this example after size reduction is approximately 1.04. Additionally, the particles after size reduction had a Brunauer, Emmett and Teller (BET) specific surface area of approximately 40 m2/g to 50 m2/g.

The purity of the aggregates produced in this sample were measured using mass spectrometry and x ray fluorescence (XRF) spectroscopy. The ratio of carbon to other elements, except for hydrogen, measured in 16 different batches was from 99.86% to 99.98%, with an average of 99.94% carbon.

In this example, carbon nanoparticles were generated using a thermal hot-wire processing system. The precursor material was methane, which was flowed from 1 slm to 5 slm. With these flow rates and the tool geometry, the resonance time of the gas in the reaction chamber was from approximately 20 second to 30 seconds, and the carbon particle production rate was from approximately 20 g/hr.

Further details pertaining to such a processing system can be found in the previously mentioned U.S. Pat. No. 9,862,602, titled “CRACKING OF A PROCESS GAS.”

FIG. 7G, FIG. 7H and FIG. 7I show TEM images of as synthesized carbon nanoparticles of this example. The carbon nanoparticles contain connected multi-walled spherical fullerenes (MWSFs) with layers of graphene coating the connected MWSFs. The ratio of multi-walled fullerenes to graphene allotropes in this example is approximately 30% due to the relatively long resonance times allowing thicker, or more, layers of graphene to coat the MWSFs. No catalyst was used in this process, and therefore, there is no central seed containing contaminants. The as synthesized aggregate particles produced in this example had particle sizes of approximately 10 μm to 500 μm. FIG. 7J shows a Raman spectrum from the aggregates of this example. The Raman signature of the as synthesized particles in this example is indicative of the thicker graphene layers which coat the MWSFs in the as synthesized material. Additionally, the as synthesized particles had a Brunauer, Emmett and Teller (BET) specific surface area of approximately 90 m2/g to 100 m2/g.

FIG. 7K and FIG. 7L show TEM images of the carbon nanoparticles of this example. Specifically, the images depict the carbon nanoparticles after performance of size reduction by grinding in a ball mill. The size reduction process conditions were the same as those described as pertains to the foregoing FIG. 7G through FIG. 7J. After size reduction, the aggregate particles produced in this example had a particle size of approximately 1 μm to 5 μm. The TEM images show that the connected MWSFs that were buried in the graphene coating can be observed after size reduction. FIG. 7M shows a Raman spectrum from the aggregates of this example after size reduction taken with 532 nm incident light. The ID/IG for the aggregate particles in this example after size reduction is approximately 1, indicating that the connected MWSFs that were buried in the graphene coating as synthesized had become detectable in Raman after size reduction, and were well ordered. The particles after size reduction had a Brunauer, Emmett and Teller (BET) specific surface area of approximately 90 m2/g to 100 m2/g.

FIG. 7N is a scanning electron microscope (SEM) image of carbon aggregates showing the graphite and graphene allotropes at a first magnification. FIG. 7O is a SEM image of carbon aggregates showing the graphite and graphene allotropes at a second magnification. The layered graphene is clearly shown within the distortion (wrinkles) of the carbon. The 3D structure of the carbon allotropes is also visible.

The particle size distribution of the carbon particles of FIG. 7N and FIG. 7O is shown in FIG. 7P. The mass basis cumulative particle size distribution 706 corresponds to the left y axis in the graph (Q3(x) [%]). The histogram of the mass particle size distribution 708 corresponds to the right axis in the graph (dQ3(x) [%]). The median particle size is approximately 33 μm. The 10th percentile particle size is approximately 9 μm, and the 90th percentile particle size is approximately 103 μm. The mass density of the particles is approximately 10 g/L.

The particle size distribution of the carbon particles captured from a multiple-stage reactor is shown in FIG. 7Q. The mass basis cumulative particle size distribution 714 corresponds to the left y axis in the graph (Q3(x) [%]). The histogram of the mass particle size distribution 716 corresponds to the right axis in the graph (dQ3(x) [%]). The median particle size captured is approximately 11 μm. The 10th percentile particle size is approximately 3.5 μm, and the 90th percentile particle size is approximately 21 μm. The graph in FIG. 7Q also shows the number basis cumulative particle size distribution 718 corresponding to the left y axis in the graph (Q0(x) [%]). The median particle size by number basis is from approximately 0.1 μm to approximately 0.2 μm. The mass density of the particles collected is approximately 22 g/L.

Returning to the discussion of FIG. 7P, the graph also shows a second set of example results. Specifically, in this example, the particles were size-reduced by mechanical grinding, and then the size-reduced particles were processed using a cyclone separator. The mass basis cumulative particle size distribution 710 of the size-reduced carbon particles captured in this example corresponds to the left y axis in the graph (Q3(x) [%]). The histogram of the mass basis particle size distribution 712 corresponds to the right axis in the graph (dQ3(x) [%]). The median particle size of the size-reduced carbon particles captured in this example is approximately 6 μm. The 10th percentile particle size is from 1 μm to 2 μm, and the 90th percentile particle size is from 10 μm to 20 μm.

Further details pertaining to making and using cyclone separators can be found in U.S. patent application Ser. No. 15/725,928, filed Oct. 5, 2017, titled “MICROWAVE REACTOR SYSTEM WITH GAS-SOLIDS SEPARATION”, which is hereby incorporated by reference in its entirety.

### High Purity Carbon Allotropes Produced Using Microwave Reactor Systems

In some cases, carbon particles and aggregates containing graphite, graphene and amorphous carbon can be generated using a microwave plasma reactor system using a precursor material that contains methane, or contains isopropyl alcohol (IPA), or contains ethanol, or contains a condensed hydrocarbon (e.g., hexane). In some other examples, the carbon-containing precursors are optionally mixed with a supply gas (e.g., argon). The particles produced in this example contained graphite, graphene, amorphous carbon and no seed particles. The particles in this example had a ratio of carbon to other elements (other than hydrogen) of approximately 99.5% or greater.

In one particular example, a hydrocarbon was the input material for the microwave plasma reactor, and the separated outputs of the reactor comprised hydrogen gas and carbon particles containing graphite, graphene and amorphous carbon. The carbon particles were separated from the hydrogen gas in a multi-stage gas-solid separation system. The solids loading of the separated outputs from the reactor was from 0.001 g/L to 2.5 g/L.

FIG. 7R, FIG. 7S, and FIG. 7T are TEM images of as synthesized carbon nanoparticles. The images show examples of graphite, graphene and amorphous carbon allotropes. The layers of graphene and other carbon materials can be clearly seen in the images.

The particle size distribution of the carbon particles captured is shown in FIG. 7U. The mass basis cumulative particle size distribution 720 corresponds to the left y axis in the graph (Q3(x) [%]). The histogram of the mass particle size distribution 722 corresponds to the right axis in the graph (dQ3(x) [%]). The median particle size captured in the cyclone separator in this example was approximately 14 μm. The 10th percentile particle size was approximately 5 μm, and the 90th percentile particle size was approximately 28 μm. The graph in FIG. 7U also shows the number basis cumulative particle size distribution 724 corresponding to the left y axis in the graph (Q0(x) [%]). The median particle size by number basis in this example was from approximately 0.1 μm to approximately 0.2 μm.

FIG. 7V, FIG. 7W, and FIGS. 7X, and 7Y are images that show three-dimensional carbon-containing structures that are grown onto other three-dimensional structures. FIG. 7V is a 100× magnification of three-dimensional carbon structures grown onto carbon fibers, whereas FIG. 7W is a 200× magnification of three-dimensional carbon structures grown onto carbon fibers. FIG. 7X is a 10000× magnification of three-dimensional carbon structures grown onto carbon fibers. The three-dimensional carbon growth over the fiber surface is shown. FIG. 7Y is a 10000× magnification of three-dimensional carbon structures grown onto carbon fibers. The image depicts growth onto the basal plane as well as onto edge planes.

More specifically, FIGS. 7V-7Y show example SEM images of 3D carbon materials grown onto fibers using plasma energy from a microwave plasma reactor as well as thermal energy from a thermal reactor. FIG. 7V shows an SEM image of intersecting fibers 731 and 732 with 3D carbon growth 730 grown on the surface of the fibers. FIG. 7W is a higher magnification image (the scale bar is 300 □m compared to 500 □m for FIG. 7V) showing 3D carbon growth 730 on the fiber 732. FIG. 7X is a further magnified view (scale bar is 40 m) showing 3D carbon growth 730 on fiber surface 735, where the 3D nature of the carbon growth 730 can be clearly seen. FIG. 7Y shows a close-up view (scale bar is 500 nm) of the carbon alone, showing interconnection between basal planes 736 and edge planes 734 of numerous sub-particles of the 3D carbon material grown on the fiber. FIGS. 7V-7Y demonstrate the ability to grow 3D carbon on a 3D fiber structure according to some embodiments, such as 3D carbon growth grown on a 3D carbon fiber.

In some embodiments, 3D carbon growth on fibers can be achieved by introducing a plurality of fibers into the microwave plasma reactor and using plasma in the microwave reactor to etch the fibers. The etching creates nucleation sites such that when carbon particles and sub-particles are created by hydrocarbon disassociation in the reactor, growth of 3D carbon structures is initiated at these nucleation sites. The direct growth of the 3D carbon structures on the fibers, which themselves are three-dimensional in nature, provides a highly integrated, 3D structure with pores into which resin can permeate. This 3D reinforcement matrix (including the 3D carbon structures integrated with high aspect ratio reinforcing fibers) for a resin composite results in enhanced material properties, such as tensile strength and shear, compared to composites with conventional fibers that have smooth surfaces and which smooth surfaces typically delaminate from the resin matrix.

### Functionalizing Carbon

In some embodiments, carbon materials, such as 3D carbon materials described herein, can be functionalized to promote adhesion and/or add elements such as oxygen, nitrogen, carbon, silicon, or hardening agents. In some embodiments, the carbon materials can be functionalized in situ—that is, within the same reactor in which the carbon materials are produced. In some embodiments, the carbon materials can be functionalized in post-processing. For example, the surfaces of fullerenes or graphene can be functionalized with oxygen- or nitrogen-containing species which form bonds with polymers of the resin matrix, thus improving adhesion and providing strong binding to enhance the strength of composites.

Embodiments include functionalizing surface treatments for carbon (e.g., CNTs, CNO, graphene, 3D carbon materials such as 3D graphene) utilizing plasma reactors (e.g., microwave plasma reactors) described herein. Various embodiments can include in situ surface treatment during creation of carbon materials that can be combined with a binder or polymer in a composite material. Various embodiments can include surface treatment after creation of the carbon materials while the carbon materials are still within the reactor.

In the foregoing specification, the disclosure has been described with reference to specific implementations thereof. It will however be evident that various modifications and changes may be made thereto without departing from the broader spirit and scope of the disclosure. For example, the above-described process flows are described with reference to an ordering of process actions. However, the ordering of many of the described process actions may be changed without affecting the scope or operation of the disclosure. The specification and drawings are to be regarded in an illustrative sense rather than in a restrictive sense.

What is claimed is:

1. A component, comprising:
a material; and
at least one resonant sensor embedded within the material, wherein the at least one resonant sensor is formed from a composite material,
wherein the at least resonant sensor undergoes a change of permittivity responsive to metabolic activity of a microorganism.

2. The component of claim 1, wherein the material is a blood culture medium.

3. The component of claim 2, wherein the blood culture medium is contained within a blood culture bottle.

4. The component of claim 3, wherein the at least one resonant sensor is embedded within a wall of the blood culture bottle or the at least one resonant sensor is embedded in an adhesive or label affixed to an exterior wall of the blood culture bottle.

5. The component of claim 1, wherein the change in permittivity is due to production of carbon dioxide by the microorganism.

6. The component of claim 1, wherein the composite material comprises a carbonaceous material.

7. The component of claim 6, wherein the carbonaceous material comprises graphene.

8. The component of claim 1, further comprising a processor configured to analyze the change in permittivity to determine antimicrobial susceptibility of the microorganism.

9. The component of claim 8, wherein the processor is further configured to generate a report indicating antimicrobial susceptibility results.

10. The component of claim 1, wherein the at least one resonant sensor is configured to resonate at a first frequency in response to an electromagnetic stimulus when the material is in a first state.

11. The component of claim 10, wherein the at least one resonant sensor is configured to resonate at a second frequency in response to the electromagnetic stimulus when the material is in a second state.

12. The component of claim 11, wherein the first state corresponds to an absence of the microorganism and the second state corresponds to a presence of the microorganism.

13. The component of claim 1, wherein the at least one resonant sensor comprises a plurality of resonant sensors arranged in an array.

14. The component of claim 13, wherein each resonant sensor in the array is configured to detect changes in permittivity in a different region of the material.

15. The component of claim 1, further comprising an electromagnetic stimulus source configured to emit an electromagnetic signal to stimulate the at least one resonant sensor.

16. The component of claim 15, wherein the electromagnetic stimulus source is configured to emit a chirp signal spanning a range of frequencies.

17. The component of claim 1, further comprising a detector configured to measure changes in at least one of amplitude and frequency of a resonant response of the at least one resonant sensor.

18. The component of claim 1, wherein the at least one resonant sensor is configured to detect changes in permittivity due to changes in pH of the material.

19. The component of claim 1, wherein the component is part of an antimicrobial susceptibility testing system comprising multiple wells, each well containing a different antimicrobial agent.

20. The component of claim 19, wherein the at least one resonant sensor is configured to detect changes in permittivity in each well to determine effectiveness of different antimicrobial agents against the microorganism.

21. A component, comprising:
a material; and
at least one resonant sensor embedded within the material, wherein the at least one resonant sensor is formed from a composite material,
wherein the at least resonant sensor undergoes a change of permeability responsive to metabolic activity of a microorganism.

22. The component of claim 21, wherein the at least one resonant sensor is embedded within an adhesive or label.
